(19) United States
(12) Patent Application Publication
(10) Pub. No.: US 2025/0075241 A1
(43) Pub. Date: Mar. 6, 2025

(54) PROTECTION OF LINEAR DEOXYRIBONUCLEIC ACID FROM EXONUCLEOLYTIC DEGRADATION

(71) Applicant: The Governing Council of the University of Toronto, Toronto (CA)

(72) Inventors: Keith Ian PARDEE, Toronto (CA); Masoud NOROUZI, Toronto (CA)

(73) Assignee: The Governing Council of the University of Toronto, Toronto, ON (CA)

(21) Appl. No.: 18/274,161

(22) PCT Filed: Jan. 27, 2022
(86) PCT No.: PCT/CA2022/050113

§ 371 Date: Jul. 25, 2023

Related U.S. Application Data
(60) Provisional application No. 63/142,097, filed on Jan. 27, 2021.

(51) Int. Cl. C12P 21/02; C12N 9/12; C12N 15/11

(52) U.S. Cl. CPC C12P 21/02; C12N 9/1247; C12N 15/11; C12N 2310/3513; C12Y 207/07006

(57) ABSTRACT

A linear double stranded deoxyribonucleic acid (dsDNA) molecule comprising operatively linked in 5′ to 3′ direction: a) one or more Ter sites at 5′ terminus (“5′ Ter”); b) a segment comprising a DNA sequence of interest; and c) one or more Ter sites at 3′ terminus (“3′ Ter). Also methods of protecting DNA sequences of interest from exonuclease degradation using the DNA constructs of the present disclosure, cells transformed with the DNA construct of the present disclosure and cell-free synthetic biology system comprising a linear dsDNA molecule of the present disclosure.

### FIELD OF THE TECHNOLOGY

The present disclosure relates to the protection of linear deoxyribonucleic acid (DNA) molecules from exonucleolytic degradation in a biology environment and to methods of producing proteins from linear expression templates.

### BACKGROUND

Over the past two decades cell-free synthetic biology has established itself as a versatile platform for advancing biological research at a pace unattainable by traditional cell-based methods. From in vitro expression of proteins for structural [1,2] and microarray analyses, [3] to rapid prototyping of enzymatic [4,5] and regulatory elements, [6-8] to field-deployable diagnostics, [9] and even industrial-scale biomanufacturing of therapeutics, cell-free systems (CFSs) have revolutionised the discovery process across many research disciplines. These CFSs typically consist of extracted cellular transcription-translation (TXTL) machinery in a mixture with energy regeneration and biosynthesis micro components. The great majority of these applications are reliant on Escherichia coli (E. coli) as their chassis organism for extract preparation. [11] Although, particularly in recent years, Vibrio natriegens (V. nat) has also been championed by multiple research laboratories as the next-generation chassis organism due to its faster growth cycle and desirable biosafety level. [12-16]

These bacterial CFSs are known to rely strictly on circular DNA input, as linear expression templates (LETs) with free termini are highly susceptible to exonucleolytic degradation by endogenous enzymes present in the extracts. [17] In E. coli, the helicase exonuclease complex RecBCD has been well-reported to degrade linear DNA with high processivity. [18] Endogenous exonucleases in V. nat extracts are similarly capable of degrading LETs. [16,19] Therefore, despite being extremely useful and advantageous compared to in vivo techniques, this incompatibility with linear DNA means that the huge potential of CFSs is yet to be fully tapped. For instance, cloning and preparation of plasmid templates for a gene of interest can take a minimum of two days by standard laboratory practices; whereas the same gene could be prepared as a LET by Polymerase Chain Reaction (PCR) within three hours and at a far higher throughput. Therefore, by simply replacing plasmids with LETs in cell-free applications a considerable amount of time, cost and labour could be spared to accelerate and expand the discovery process in both fundamental and applied research.[20]

Recognising this potential, various research laboratories have so far tried to develop methods to protect LETs in bacterial extracts.[2,17,19,21-25] In E. coli CFSs, one of the most effective and widely used solutions is the Lambda GamS protein which specifically inhibits RecBCD thereby prolonging the lifetime of LETs in the extract. However, despite some recent efforts,[16, 19,25] the same techniques that work for E. coli are either ineffective or poorly functional in V. nat extracts-likely due to its divergent DNA degradation mechanisms.[14]

The “Tus-Ter” E. coli DNA replication termination system, which has homologues across many γ-proteobacterial strains [28], involves a protein module—the “Tus” protein, and a 23 base pair cognate DNA sequence module—the “Ter” sequence, with a remarkable equilibrium binding constant (KD) of 3.4×10⁻¹³ M.²⁸ The high-affinity binding of Tus to the Ter sequence strongly inhibits the progress of helicase-containing complexes towards any DNA sequence preceding the Ter site [29,30] even in eukaryotic systems [31]. As such, the Tus-Ter system has been proposed as a system to regulate replication fork arrest and can be utilized for disrupting DNA replication.

### SUMMARY

In one embodiment, the present disclosure is a linear double stranded deoxyribonucleic acid (dsDNA) molecule comprising operatively linked in the 5′ to 3′ direction: a) one or more Ter sites at the 5′ terminus (“5′ Ter”); b) a segment comprising DNA sequence of interest; and c) one or more Ter sites at the 3′ terminus (“3′ Ter).

In one embodiment of the linear dsDNA molecule, the DNA sequence of interest is a functional DNA sequence.

In another embodiment of the linear dsDNA molecule, the functional DNA sequence is a gene, a regulatory sequence, a splice site a binding site, a primer, an aptamer or combinations thereof.

In another embodiment of the linear dsDNA molecule, the 3′ Ter is downstream a terminator sequence.

In another embodiment of the linear dsDNA molecule, the DNA sequence of interest is a coding sequence for encoding an expression product and the terminator sequence is located after a STOP codon of the DNA coding sequence and before the 3′ Ter.

In another embodiment of the linear dsDNA molecule, the linear dsDNA molecule further comprises a 5′ DNA buffer region upstream the 5′ end of the DNA sequence of interest and a 3′ DNA buffer region 3′ end downstream the DNA sequence of interest, and wherein the 5′ DNA buffer region includes between 0 to 300 base pairs and the 3′ DNA buffer region includes between 0 to 125 base pairs.

In another embodiment of the linear dsDNA molecule, the DNA sequence of interest is the coding sequence as defined in claim 5, and wherein the 3′ DNA buffer ranges between 45 and 125 base pairs.

In another embodiment of the linear dsDNA molecule, the linear dsDNA further comprises a Tus protein bound to the 5′ Ter site and another Tus protein bound to the 3′ Ter.

In another embodiment of the linear dsDNA molecule, at least one of the one or more Ter sites comprises SEQ ID NO:1.

In another embodiment of the linear dsDNA molecule, the one or more Ter sites at the 5′ terminus comprises SEQ ID NO: 2 and the one or more Ter sites at the 3′ terminus comprises SEQ ID NO: 3.

In another embodiment, the present disclosure relates to a method of protecting a linear deoxyribonucleic acid (DNA) molecule having a free 5′ terminus and a free 3′ terminus from exonuclease degradation comprising: a) adding one or more Ter sites at the free 5′ terminus (“5′ Ter) of the DNA molecule and adding one or more Ter sites at the 3′ terminus (“3′ Ter”) of the DNA molecule, and b) binding a Tus protein to each of the 5′ Ter and the 3′ Ter.

In one embodiment of the method of protecting the linear DNA molecule, the linear DNA molecule is a double stranded deoxyribonucleic acid (DNA) molecule.

In another embodiment of the method of protecting the linear DNA molecule, the exonuclease is a bacterial exonuclease.

In another embodiment of the method of protecting the linear DNA molecule, the DNA molecule includes a functional DNA molecule.

In another embodiment of the method of protecting the linear DNA molecule, the DNA molecule includes a gene, a regulatory sequence, a splice site a binding site, a primer, an aptamer or combinations thereof.

In another embodiment of the method of protecting the linear DNA molecule, the DNA molecule includes a terminator sequence and the 3′ Ter site is downstream the terminator sequence.

In another embodiment of the method of protecting the linear DNA molecule, the DNA molecule includes a coding sequence for encoding an expression product and the terminator sequence is located after a STOP codon of the DNA molecule coding sequence and before the 3′ Ter.

In another embodiment of the method of protecting the linear DNA molecule, the method further comprises adding a 5′ DNA buffer region upstream the 5′ end of the DNA molecule and a 3′ DNA buffer region 3′ end downstream of the DNA molecule, and wherein the 5′ DNA buffer region includes between 0 to 300 base pairs and the 3′ DNA buffer region includes between 0 to 125 base pairs.

In another embodiment of the method of protecting the linear DNA molecule, the linear DNA molecule includes the coding sequence for encoding the expression product, and wherein the 3′ DNA buffer ranges between 45 and 125 base pairs.

In another embodiment of the method of protecting the linear DNA molecule, the Tus is provided as purified Tus or as a Tus-expressing bacterial strain.

In another embodiment of the method of protecting the linear DNA molecule, the Tus is provided as a Tus-expressing bacterial strain under control of an endogenous bacterial RNA polymerase.

In another embodiment of the method of protecting the linear DNA molecule, at least one of the one or more Ter sites comprises SEQ ID NO:1.

In another embodiment of the method of protecting the linear DNA molecule, at least one of the one or more Ter sites at the 5′ terminus comprises SEQ ID NO: 2 and the one or more Ter sites at the 3′ terminus comprises SEQ ID NO: 3.

In another embodiment, the present disclosure relates to a method of synthesizing a polypeptide of interest in a cell-free protein synthesis (CFPS) reaction mixture comprising: a) providing a linear dsDNA molecule of the present disclosure, wherein the DNA sequence of interest is a coding sequence for encoding the polypeptide of interest, b) providing a Tus protein, and c) adding the linear dsDNA and the Tus protein to the CFPS, thereby synthesizing the polypeptide of interest.

In one embodiment of the method of synthesizing a polypeptide of interest in a CFPS reaction mixture, the 3′ Ter is downstream a terminator sequence.

In another embodiment of the method of synthesizing a polypeptide of interest in a CFPS reaction mixture, the terminator sequence is located after a STOP codon of the DNA coding sequence and before the 3′ Ter.

In another embodiment of the method of synthesizing a polypeptide of interest in a CFPS reaction mixture, the linear dsDNA molecule further comprises a 5′ DNA buffer region upstream the 5′ end of the DNA sequence of interest and a 3′ DNA buffer region 3′ end downstream the DNA sequence of interest, and wherein the 5′ DNA buffer region includes between 0 to 300 base pairs and the 3′ DNA buffer region includes between about 45 to about 125 base pairs.

In another embodiment of the method of synthesizing a polypeptide of interest in a CFPS reaction mixture, the Tus protein is provided as a purified Tus protein.

In another embodiment of the method of synthesizing a polypeptide of interest in a CFPS reaction mixture, the CFPS includes a bacteriophage RNA polymerase.

In another embodiment of the method of synthesizing a polypeptide of interest in a CFPS reaction mixture, the Tus protein is provided as a Tus-expressing bacterial strain.

In another embodiment of the method of synthesizing a polypeptide of interest in a CFPS reaction mixture, the CFPS is an E. coli lysate-based protein expression having endogenous E. coli RNA polymerase.

In another embodiment of the method of synthesizing a polypeptide of interest in a CFPS reaction mixture, the CFPS is derived from eukaryotes or prokaryotes.

In another embodiment of the method of synthesizing a polypeptide of interest in a CFPS reaction mixture, at least one of the one or more Ter sites comprises SEQ ID NO: 1.

In another embodiment of the method of synthesizing a polypeptide of interest in a CFPS reaction mixture, at least one of the one or more Ter sites at the 5′ terminus comprises SEQ ID NO: 2 and the one or more Ter sites at the 3′ terminus comprises SEQ ID NO: 3.

In another embodiment, the present disclosure provides for a cell transformed with a linear double stranded DNA according to any embodiment of the present disclosure. In one aspect, the cell is a bacterium.

In another embodiment, the present disclosure is a cell-free synthetic biology system comprising the linear dsDNA molecule as defined in any embodiment of the present invention.

In one embodiment, the cell-free synthetic biology system comprises an E. coli lysate, a V. natriegens lysate or a B. subtilis lysate.

In another embodiment of the cell-free synthetic biology system, the cell-free synthetic biology system is derived from eukaryotes or prokaryotes.

### BRIEF DESCRIPTION OF THE DRAWINGS

A detailed description of the preferred embodiments is provided herein below by way of example only and with reference to the following drawings, in which:

FIG. 1A. Schematic illustration of Tus-Ter protection of linear DNA. In the absence of Tus protein and terminal Ter sites, the helicase-exonucleases present in cell-free lysates can degrade the double-stranded DNA from either end. In the presence of terminal Ter sites, and Tus firmly bound to the Ter sites, the progress of helicase-exonucleases is blocked, thus protecting the linear DNA.

FIG. 1B. Native PAGE electrophoretic mobility shift assay (EMSA) for the binding of Tus protein at increasing concentrations (0-1000 nM) to terminal Ter sites on linear DNA (at a constant 5 ng/μL). Two different sequence configurations are shown; one with ×1 Ter site on each 5′/3′ terminus (×2 Ter sites in total, as in panel A in the top panel, and the other with ×2 tandem Ter sites on each 5′/3′ terminus (×4 Ter sites in total). At nonsaturating Tus concentrations, ×2 and ×4 gel shift events take place in the top and the bottom panels, respectively, as indicated by red pointers. Demonstrating a one-to-one, specific binding between Tus and each Ter site on a linear expression template.

FIG. 1C. Agarose gel analysis of the degradation profile of a Cy5-labeled linear DNA (10 nM) with terminal Ter sites under active expression in an E. coli-based lysate, in the absence (−Tus) or presence (+Tus) of Tus (5 μM) over 120 min. A 3 μL aliquot of the crude cell-free mixture is loaded in each lane.

FIG. 1D. Band intensity analysis for the agarose gels in FIG. 1C using ImageJ. As shown, linear DNA is completely degraded after 15 min in the absence of Tus, whereas in the presence of Tus, linear DNA can remain protected for at least 2 h.

FIG. 2A. (SEQ ID NOS: 123 and 124) Experimental design for PCR of linear templates. mCherry is shown as a representative gene of interest. Primers each without or with a Ter overhang (not shown) were designed on SnapGene® Viewer to bind at the indicated locations upstream of T7 promoter and downstream of stop codon. In this manner, PCR can be performed to amplify the gene of interest using any Forward-Reverse primer combination to produce LETs with varying buffer region lengths and sequences.

FIG. 2B. Schematic representation of an ideal LET design for use with the Tus-Ter system. 5′ and 3′ zero positions are marked with arrows. At the 5′ end, plasmid-level LET expression can be restored with as few as a 0 bp buffer sequence. At the 3′ end a T7 terminator sequence (48 bp) is used for more effective Tus binding, and plasmid-level LET expression would require at least a terminator sequence, in an example, a 48 bp buffer sequence. At both the 5′ and 3′ termini, and especially at the 3′ terminus, an about 125 bp buffer sequence will be sufficient for effective LET productivity. However, less and more than 125 bp may be used. RBS: ribosome binding site. It should be understood that even without a terminator sequence substantial protection of the LET is achieved. A terminator sequence may not be included for example when the constructs of the present disclosure are used to protect DNA sequences that do not transcribe. Similarly, a terminator sequence may not be required for analytical applications where high/plasmid-level protein expression is not essential.

FIGS. 3A-3D. Tus-Ter protection of linear DNA in E. coli based lysates. LETs with different buffer region lengths with (light grey=Ter-LET) or without (dark grey=LET) terminal Ter sites were added to cell-free reactions. 3A) mCherry in Lysate A at 15 hour timepoint; expression from LETs with 0-300 bp buffer on either end, or 0/10/20 bp on 5′ end and 125 bp on 3′ end, or 0/10/20 bp on either end followed by the T7 terminator on the 3′ end. Plasmid expression is shown in green. 3B) deGFP in Lysate A at 5 hour timepoint; templates are as described in 3A. 3C) mCherry in Lysate B at 12.5 hour timepoint; templates are as described in 3A. 3D) deGFP in Lysate B at 4.5 hour timepoint. Here the expression from two LETs is shown in the absence or presence of Tus. One LET with 125 bp buffer on either end and the other with 0 bp on 5′ and 125 bp on 3′ end. All measurements are the average of technical triplicates +/−SD.

FIGS. 4A to 4B. Protection of linear DNA using endogenously expressed Tus, and under endogenous transcriptional control. Equimolar amounts (10 nM) of plasmids, and LETs with (Ter-LET) or without (LET) terminal Ter sites were added to cell-free reactions. (4A) Linear vs plasmid expression comparison for mCherry in BL21-Tus (15 h time point). LETs 0-125 and TO are as described in FIG. 3. (4B) Linear vs plasmid expression comparison for deGFP in BL21-Tus (5 h time point); templates are as described in 4A. (4C) Real-time linear vs plasmid expression comparison for deGFP in Lysate A in the presence Figs of Tus, under the control of the endogenous E. coli RNA polymerase. Ter-T500-0 is a linear template with Ter sites immediately upstream of the OR2—OR1-Pr promoter and downstream of the T500 terminator. TO is a linear template starting with the OR2—OR1-Pr promoter, but ending in a T7 terminator (as opposed to T500) sequence immediately after the stop codon. LET 125 is a linear template with 125 bp buffer upstream of the OR2—OR1-Pr promoter, and downstream of the stop codon, based on the pBEST plasmid backbone. (4D) 8 h time point comparison for LETs vs Ter-LETs shown in 4C. All measurements are the average of technical triplicates±SD.

FIGS. 5A-5B. Tus-Ter protection of linear DNA in V. nat based lysates. Equimolar amounts (10 nM) of plasmids, and LETs with (Ter-LET) or without (LET) terminal Ter sites were added to cell-free reactions. (5A) Linear vs plasmid expression comparison for mCherry in a V. natriegens based lysate (15 h time point). A range of mCherry LETs with different buffer region lengths/sequences (as indicated in FIG. 3A) were tested against a plasmid template in reactions containing Tus. (5B) Linear vs plasmid expression comparison for deGFP in a V. natriegens based lysate (4 h time point). Selected LETs (from the set presented in FIG. 3B) for deGFP were tested against a plasmid template in reactions containing Tus. All measurements are the average of technical triplicates +/−SD.

FIGS. 6A-6B. Comparison between the LET protection efficiency of Tus vs GamS in E. coli and V. nat CFSs. 6A) mCherry expression in the absence (control) or presence of GamS and Tus in E. coli Lysate B. Expression from plasmid and two different Ter-LETs are shown for each condition. 6B) mCherry expression in the absence (control) or presence of GamS and Tus in a V. nat lysate. Expression from plasmid and two different Ter-LETs are shown for each condition. All measurements are the average of technical triplicates +/−SD.

FIGS. 7A-7B. Different expression dynamics for mCherry and deGFP. Expression time course for deGFP (7A) and mCherry (7B) plasmids (in E. coli Lysate A) is shown over 8 and 15 hours, respectively. Detectable signal appears at approximately 12 minutes for deGFP and 70 minutes for mCherry. Measurements are the average of three technical replicates +/−SD.

FIGS. 8A-8B. The addition of Tus or Ter on their own Does Not affect gene expression in cell-free reactions. 8A: time course data for mCherry expression from plasmids in the presence or absence of Tus. 8B: time course data for mCherry expression from LETs with 50 and 100 bp buffer sequence with (+) or without (−) Ter on both termini. Measurements are the average of three technical replicates +/−SD.

FIGS. 9A-9B. Representative image for agarose gel electrophoresis analysis of Plasmids (9A) and LET PCRs (9B) used in this disclosure.

In the drawings, one embodiment of the disclosure is illustrated by way of example. It is to be expressly understood that the description and drawings are only for the purpose of illustration and as an aid to understanding and are not intended as a definition of the limits of the disclosure.

### DETAILED DESCRIPTION

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices and materials are now described. All technical and patent publications cited herein are incorporated herein by reference in their entirety. Nothing herein is to be construed as an admission that the disclosure is not entitled to antedate such disclosure by virtue of prior disclosure.

All numerical designations, e.g., pH, temperature, time, concentration and molecular weight, including ranges, are approximations which are varied (+) or (−) by increments of 1.0 or 0.1, as appropriate, or alternatively by a variation of +/−20%, +/−15%, or alternatively +/−10%, or alternatively +/−5% or alternatively +/−2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure. Embodiments defined by each of these transition terms are within the scope of this disclosure.

“Functional DNA Sequence” is meant to include a DNA sequence that is transcribed or bound by particular proteins or RNA molecules. Non-limiting examples of functional DNA sequences include a gene, a regulatory sequence, a splice site, a binding site, primers, aptamers and so forth.

A “terminator” is a DNA sequence-based element that defines the end of a transcriptional unit (such as a gene) and initiate the process of releasing the newly synthesized RNA from the transcription machinery.

A “cell free protein synthesis (CFPS)” reaction mixture typically contains a crude or partially-purified eukaryote or bacterial (such as E. coli, V. nat., S. subtillis) extract, a DNA or RNA translation template, and a suitable reaction buffer for promoting cell-free protein synthesis from the RNA translation template. In some aspects, the CFPS reaction mixture can include exogenous RNA translation template. In other aspects, the CFPS reaction mixture can include a DNA expression template encoding an open reading frame operably linked to a promoter element for a DNA-dependent RNA polymerase. In these other aspects, the CFPS reaction mixture can also include a DNA-dependent RNA polymerase to direct transcription of an RNA translation template encoding the open reading frame. In these other aspects, additional NTP's and divalent cation cofactor can be included in the CFPS reaction mixture. A reaction mixture is referred to as complete if it contains all reagents necessary to enable the reaction, and incomplete if it contains only a subset of the necessary reagents. It will be understood by one of ordinary skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of ordinary skill in the art that reaction components are packaged separately for commercialization and that useful commercial kits may contain any subset of the reaction components of the disclosure.

The term “primer,” as used herein, refers to an oligonucleotide capable of acting as a point of initiation of DNA synthesis under suitable conditions. Such conditions include those in which synthesis of a primer extension product complementary to a nucleic acid strand is induced in the presence of four different nucleoside triphosphates and an agent for extension (for example, a DNA polymerase or reverse transcriptase) in an appropriate buffer and at a suitable temperature.

The term “promoter” refers to a cis-acting DNA sequence that directs RNA polymerase and other trans-acting transcription factors to initiate RNA transcription from the DNA template that includes the cis-acting DNA sequence.

Provided herein are linear double-stranded (ds) DNA sequences, molecules, constructs, systems and methods for linear, double-stranded DNA protection from exonuclease degradation (exonucleolytic degradation) that is highly efficient in cell free systems. The dsDNAs and methods of the present disclosure are based on the “Tus-Ter” DNA replication termination system found in bacteria, including homologues and variants of Tus and Ter across bacteria such as E. coli and many γ-proteobacterial strains [28]. The addition of the Ter sequence to linear expression templates (LETs) with free termini in the presence of Tus protein can provide potent protection of LETs from exonucleases in cell lysate-based expression systems.

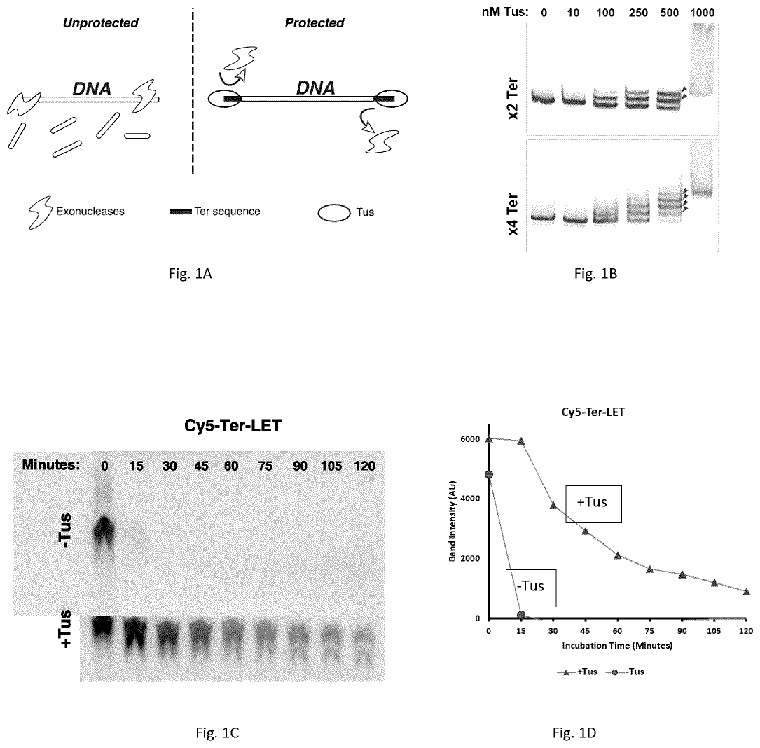

The examples below show that one or more Ter sequences appended to the 5′ and 3′ ends of a linear DNA molecule in the presence of Tus, provides protection from exonuclease degradation in cell lysate-based expression systems (FIG. 1A). Any linear DNA sequence of interest can be protected from exonuclease degradation using the dsDNAs and methods of this disclosure, including functional and non-functional DNA sequences. Ter can be oriented in the DNA constructs of the present disclosure in either permissive or non-permissive direction while still providing protection to the DNA molecule from exonuclease degradation. In the examples below, Ter is oriented so that the non-permissive face looks towards free DNA ends. However, Ter can also be flipped so that the non-permissive face looks away from the free DNA ends, while still binding to Tus and inhibiting exonucleases.

The dsDNA sequences, molecules and/or constructs of the present disclosure can be used in any lysate, extract, system, cell-free system (CFS or CFSs for plural), etc., including patient sample lysates for diagnostics that includes or is suspected to include an exonuclease.

The use case of Tus-Ter constructs to protect DNA molecules from exonuclease degradation is not limited to protein expression. Tus-Ter can be used in other applications that involve linear DNA, such as signal amplification in diagnostics, biosensing gene circuits, or DNA sequencing; where Ter sites can be incorporated as primers to protect amplified DNA from exonucleolytic degradation in the in vitro enzymatic environment. Additionally, Tus-Ter can be used to protect pre-amplified functional DNA sequences such as aptamers, aptasensors and aptazymes in an in vitro environment. The Tus-Ter constructs described herein, can provide protein expression at levels similar to or higher than plasmid-based DNA inputs. Furthermore, Tus can be provided exogenously or endogenously expressed by recombinant expression; including for example under the control of the endogenous RNA Polymerase (RNAP). The Tus-Ter systems described herein are useful in CFSs derived from eukaryotes (e.g., vertebrates, plants, insects, fungi) or prokaryotes (e.g., Escherichia coli, Vibrio natriegens, Bacillus subtilis) and the CFSs may be prepared as either purified components or semi-processed cellular extracts. CFSs can be made sterile via simple filtration, which provides for a biosafe format for use outside of the lab.

The dsDNAs of the present disclosure have many applications, such as diagnostics, DNA amplification, DNA transcription, DNA translation, and so forth.

The following examples are intended to illustrate, but not limit the disclosure.

### EXAMPLES

### Materials and Methods

### Bacterial Strains and Plasmids

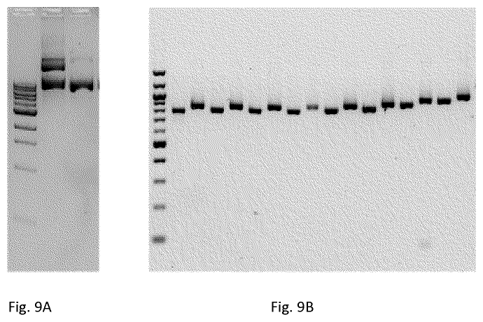

E. coli BL21 (C2530), BL21 (DE3) (C2527), 5-alafa (C2987), and SHuffle® Express (C3028) strains were purchased from NEB. V. nat (#14048) was purchased from ATCC. For plasmid construction, NEBuilder® HiFi DNA Assembly Master Mix (E2621) and standard molecular cloning procedures were used. pET24b-NusA-Tus, pET24b-mCherry and pET24b-deGFP were constructed based on the pET24b backbone from Addgene (#111702). pQE-P_{Llaco}-T7 was constructed by replacing the T5 promoter in in pQE-T7911 (a kind gift from Prof Ben Luisi's laboratory (Cambridge, UK) and originally provided by Dr. Thomas Shrader (Albert Einstein College of Medicine, NY)) for P_{LlacO-1} promoter. For use as cell-free expression template, plasmids were propagated in 5-alfa cells and purified using E.Z.N.A.® Plasmid Midi Kit (D6904-03) from Omega Bio-Tek; and further concentrated using Amicon Ultra centrifugal filter units (Z648035) from Millipore Sigma. Plasmids were eluted in nuclease-free water and quantified on a Thermo Scientific™ NanoDrop™ One UV-Vis Spectrophotometer. In all cases, A260/280 and A260/A230 ratios were 1.8-1.85 and 2.1-2.3, respectively, indicating high purity. Additionally, agarose gel electrophoresis was used to confirm plasmid quality (FIG. 9A). All coding sequences are provided in the Sequence Listing below.

### PCR for Linear Expression Templates

Q5® High-Fidelity DNA Polymerase (NEB M0491) was used for all PCRs. Primers were designed manually, checked on the SnapGene® Viewer Software, and synthesised by Eurofins Genomics or Integrated DNA Technologies. PCR reactions were all assembled in 100 μl volumes and contained 1×Q5® reaction buffer, 200 UM dNTPs, 500 nM each of forward and reverse primers, 1-10 ng of plasmid template, and 1 μl of Q5® Polymerase. PCRs were performed on an Applied Biosystems ProFlex™ thermocycler using the following conditions: 1 minute initial denaturation at 98° C.; followed by ×35 cycles of 6 second at 98° C., 15 seconds at 60° C., and 90 seconds at 72° C. After completion, all PCR products were subjected to DpnI (NEB R0176) digestion to ensure no plasmid template carryover. QIAquick PCR Purification Kit (Qiagen #28106) was used to purify DpnI digested PCR products, with final elution in 35 μl of nuclease-free water. PCR products' quantity and quality were checked as described above for plasmids, (see FIG. 9B for example gel). All primer sequences are provided in the Sequence Listing below.

### Cell-Free Lysate Preparation

E. coli based lysates were prepared essentially as described in Levine et al [32]. V. nat based lysates were prepared according to the guidelines set in Des Soyes et al [14]; and essentially following the protocols described in Levine et al with these modifications: Brain heart infusion (BHI) media containing v2 salts (204 mM NaCl, 4.2 mM KCl, 23.14 mM MgCl₂) was used for cell growth and cells were harvested at OD₆₀₀ of 7.

### Cell-Free Reaction Set-Up

Cell-free reactions' composition were based essentially on the protocols described previously with the following modifications: Phosphoenolpyruvate in the Solution B was replaced with 3-Phosphoglyceric acid, and Solution A did not contain putrescine; 20 amino acids were omitted from Solution B and added separately at a final concentration of 2.1 mM. The recipe for 20 amino acid (Sigma-Aldrich LAA21) stock solutions was adapted from with the following modifications: Arg was dissolved in ultra-pure water; and Asp, Glu, His and Tyr were dissolved in 3 M hydrochloric acid. The final concentration of T7 RNA polymerase and Tus protein were always set at 1.2 μM and 5 μM, respectively.

Purified Tus was not added to BL21-Tus lysate based reactions. Reactions with the endogenous E. coli RNAP were performed in Lysate A and supplemented with 0.05 units/μL of E. coli RNAP (NEB M0551). Where indicated, GamS (Arbor Biosciences #501024) was also added at 5 μM final concentration. Extract:total-reaction ratios were set as follows: 33% v/v for E. coli BL21, and 25% v/v for E. coli SHuffle® Express and V. nat. Cell-free reactions for each lysate were always assembled on ice as a master mix containing all components barring DNA templates, then thoroughly mixed and aliquoted so that the final volume of individual reactions was 20 μl after DNA addition. For plate reader measurements, each 20 μl reaction was immediately divided (on ice) into triplicate 6 μl volumes in a 386 Corning® microwell plate, sealed with a clear film (SARSTEDT 95.1994), and placed in a Synergy Neo2 plate reader (BioTek®). Reaction temperature was always set to 30° C., and fluorescence measurement settings were as follows: for mCherry, excitation at 587/10 nm and emission at 610/10 nm; and for deGFP, excitation at 488/9 nm and emission at 507/9 nm.

### Protein Purification

TUS: pET24b-NusA-Tus was transformed into BL21 (DE3) cells and used to inoculate an overnight Luria-Bertani (LB) culture growing at 37° C. The overnight culture was then diluted 1/200 into 1 L of fresh LB and incubated at 37° C. with shaking at 250 RPM until the cells reached mid-exponential phase (OD₆₀₀=˜0.6). Tus expression was induced by the addition of 0.5 mM Isopropyl β-D-1-thiogalactopyranoside (IPTG) and cells were grown for an additional four hours at 37° C. with shaking at 250 RPM. Cells were then harvested by centrifugation at 8000 RCF for 15 minutes, saving the pellet. For protein purification, the cell pellet was resuspended in 20 ml of ion matrix affinity chromatography (IMAC) binding buffer (50 mM Tris-HCL (PH 7.8), 300 mM NaCl, one cOmplete™ EDTA-free Protease Inhibitor tablet) and subjected to sonication on a Fisherbrand™ Q700 sonicator with the following settings: 50% amplitude, 5 seconds ON for a total of 6 minutes with 10 second OFF cycles. The cell lysate was then centrifuged at 20,000 RCF for 1 hour at 4° C., and the supernatant was passed through a 0.2 μm Basix™ syringe filter. Using an ÄKTA Pure System (Cytiva), the cleared lysate was then passed through a 5 ml HisTrap FF IMAC column (Cytiva) and eluted in binding buffer containing 500 mM imidazole (without protease inhibitor tablet).

IMAC elution fractions were then pooled and subjected to TEV cleavage for 15 hours at room temperature to remove the NusA tag. The cleaved sample was then further purified using the ÄKTA Pure System on a HiLoad® 16/600 Superdex® 75 pg gel filtration column (Cytiva) equilibrated in Tus storage buffer (50 mM Tris-HCL (PH 7.8), 300 mM NaCl, 1 mM DTT). Final protein concentration was then determined using the molar extinction coefficient of Tus (ε=39420 M⁻¹ cm⁻¹) on a Thermo Scientific™ NanoDrop™ One UV-Vis Spectrophotometer, and aliquots were flash frozen and stored at −80° C.

T7 RNA polymerase: a similar protocol to Tus was used with the following changes: E. coli BL21 cells were used for protein expression, IMAC binding buffer contained 50 mM HEPES (PH 7.5), 300 mM NaCl, 1 mM DTT, 1 mM EDTA, 20 mM imidazole, and one cOmplete™ EDTA-free Protease Inhibitor tablet; IMAC elution buffer was Binding buffer with 0.5 M imidazole and no protease inhibitor tablet; for gel filtration a HiLoad® 16/600 Superdex® 200 pg column equilibrated in T7 storage buffer (20 mM KH₂PO₄ (PH 7.5), 100 mM NaCl, 1 mM DTT, 1 mM EDTA, 0.05% Triton X-100) was used; and the molar extinction coefficient of T7 RNA polymerase was (ε=140260 M⁻¹ cm⁻¹).

### Results

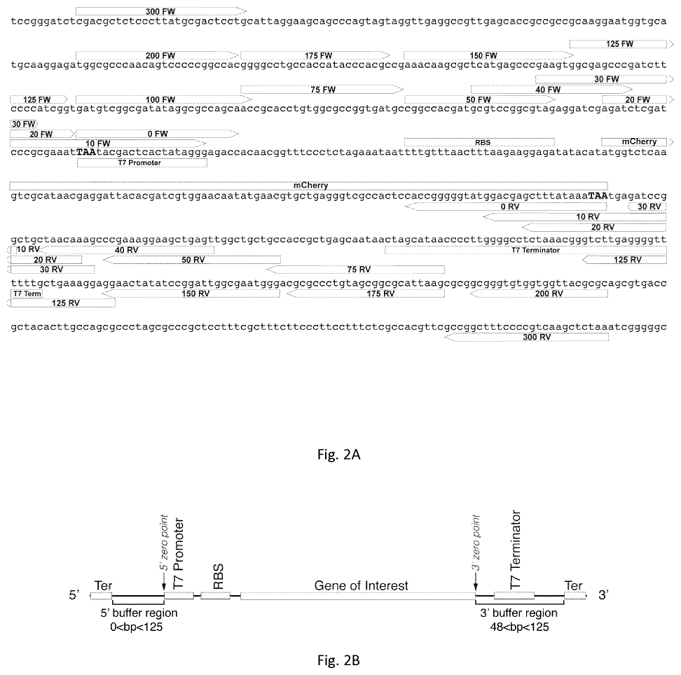

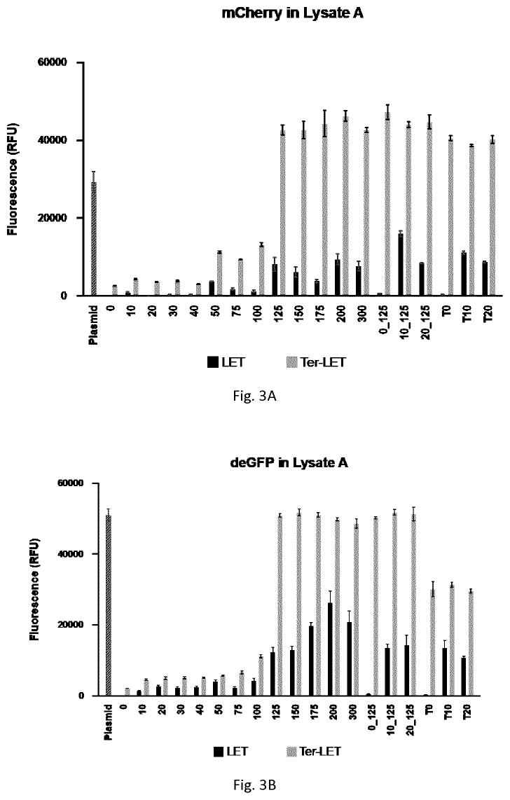

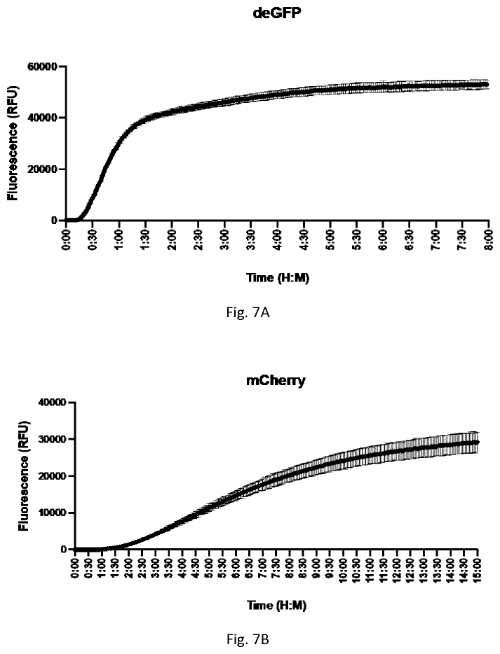

To demonstrate the effectiveness of the “Tus-Ter” protection strategy in CFSs we compared the expression levels of equimolar amounts of linear vs plasmid templates for mCherry and deGFP in E. coli and V. nat extracts. mCherry and deGFP have differing expression dynamics, with detectable signal appearing at approximately 70 and 12 minutes for mCherry (FIG. 7B) and deGFP plasmids, respectively (FIG. 7A). Thus, by including both genes in our experiments we could provide a broader representation with respect to Tus-Ter protection efficiency of target templates. Previous strategies have been shown to be more efficient in presence of long buffer regions either side of the coding frame^{19,25,32}. We therefore used specific primers to PCR amplify a range of LETs each with and without Ter, with buffer regions ranging from 0-300 bp upstream of T7 promoter and downstream of stop codon (FIG. 2). This would delineate minimal buffer sequence requirements for maximal Tus-Ter effectiveness, which in turn allows for setting forth practicable guidelines for LET design e.g. considering commercial primer and/or gene synthesis length limitations. In our laboratory, we have seen differing levels of nuclease activity from batch to batch for E. coli extracts. Therefore, for broader representation, we included two E. coli extract batches, one (BL21-based) with moderate (81% for mCherry and 75% for deGFP) and one (SHuffle® Express-based) with high (98% for both mCherry and deGFP) exonuclease activity (see FIG. 1). The inclusion of a V. nat extract was to demonstrate the robustness and orthogonality of Tus-Ter LET protection strategy with regard to different chassis organisms; as previous attempts to extend E. coli compatible methods to V. nat have not had much success^{16,19}.

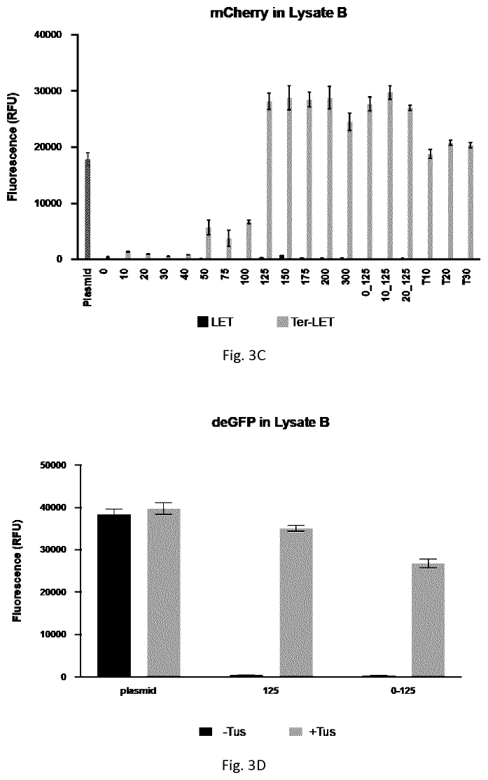

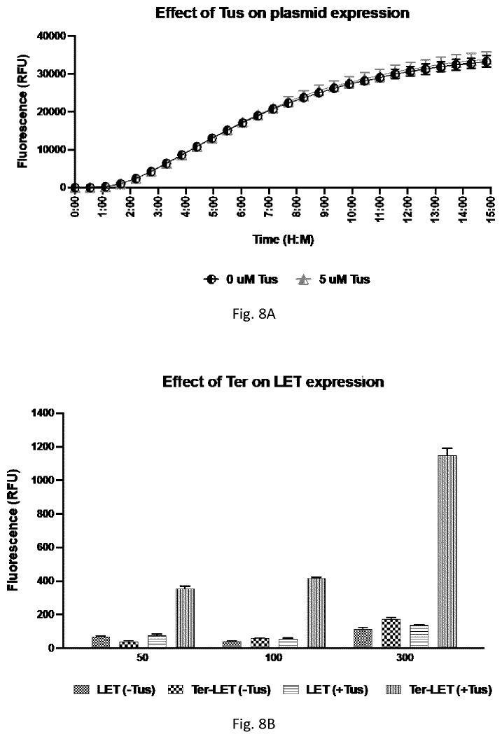

For simple implementation, we included the purified Tus protein as an additive during reaction set up and incorporated the Ter sequence as a primer extension during PCR, without the need for Tus-LET preincubation. The addition of Tus or Ter per se does not have any detectable effect on the performance of CFSs (FIGS. 8A-8B), other than the effect from the added buffer sequence in the case of Ter. Initial experiments in E. coli suggested that the presence of T7 terminator after the stop codon and preceding the Ter site is critical for efficient LET protection, as the incoming T7 polymerase can temporarily dislodge Tus and expose the 3′ terminus [34]. This is readily evident by comparing the expression levels of all LET constructs with buffer regions shorter and longer than 125 bp, position 125 falling right after the T7 terminator at 3′ (see FIG. 2). As seen in FIG. 3, beyond 125 bp the signal almost reaches saturation for both mCherry and deGFP, accentuating the role of T7 terminator in maintaining effective Tus-Ter interaction. This is in addition to the RNA stabilization effect that T7 terminator confers to RNA transcripts in CFSs [35]. Based on this observation, we hypothesized that the length of the 5′ buffer region may not be as critical as 3′. We therefore prepared LET constructs with varying 5′ buffer lengths (0-20 bp) but maintaining the 125 base pair buffer in the 3′ end. As expected, the results showed that on the 5′ end the Ter site can be appended immediately upstream of the T7 promoter with minimal effect on protection efficiency (FIG. 1). We additionally explored if shortening the 3′ buffer between the stop codon and the T7 terminator-Ter can still maintain high-level LET protection. Indeed, with these minimal-buffer LETs, mCherry linear/plasmid expression levels remained high in both of our E. coli lysates with >130% for lysate A and >100% for lysate B (FIGS. 3A and 3C). Interestingly, we also observed a stark difference in protected-linear vs plasmid expression ratio between mCherry and deGFP in E. coli; with mCherry LETs reaching up to 146% of plasmid expression levels as opposed to just 100% for corresponding deGFP LETs in Lysate A (FIGS. 3A and 3B). The exact reason for this discrepancy warrants more investigation, but it has been previously shown that gene expression regulatory processes in E. coli CFSs can affect linear and plasmid templates differently [23,36].

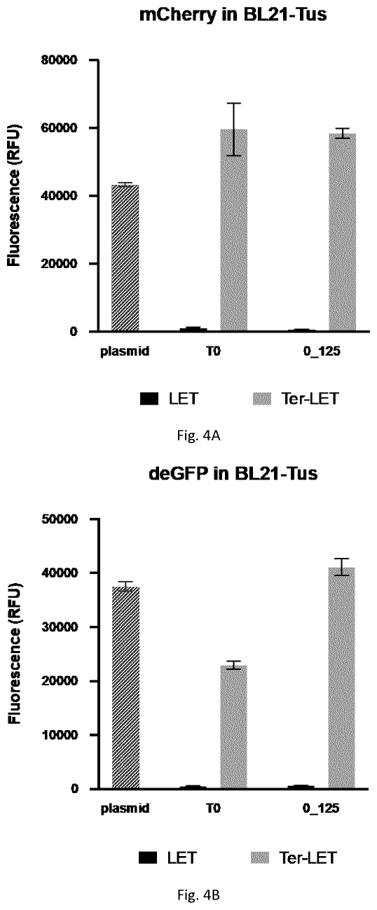

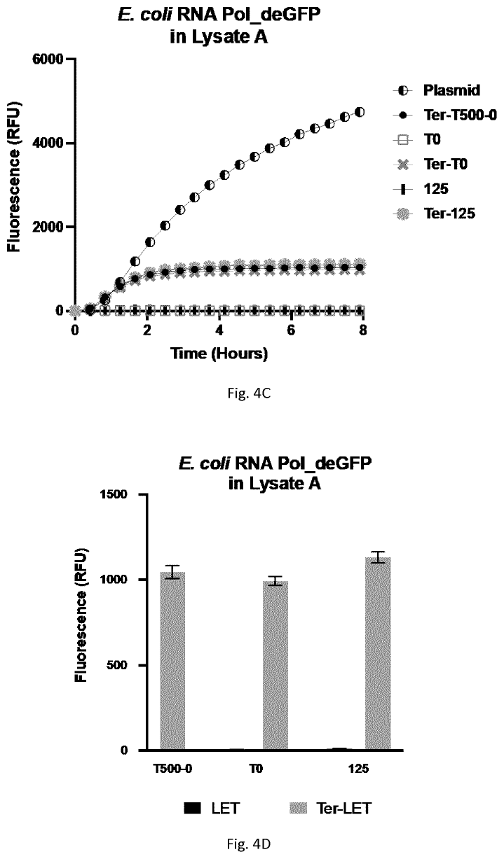

Although the addition of defined amounts of purified Tus can allow for more control over individual experiments, using a Tus-expressing bacterial strain for lysate preparation can simplify the workflow in many CFS applications. We therefore tested the expression of selected LETs (TO and 0-125) in a Tus-expressing BL21-based lysate (BL21-Tus). As seen in FIGS. 4A and 4B, in the BL21-Tus lysate, both mCherry and deGFP LETs show a very similar linear versus plasmid expression profile to the BL21+purified Tus lysate in FIGS. 3A and 3B. With this information in hand, users have a simple and low burden method for the use of linear DNA templates directly in lysate-based CFSs.

Cell-free gene expression under the control of endogenous RNA polymerases has been proven as a versatile tool for construction and characterization of synthetic gene circuits in recent years [8,36] due to the rich repertoire of endogenous transcription regulatory elements and the closer correlation between the in vitro and in vivo behavior of gene circuits under endogenous control. To demonstrate that the Tus-Ter system presented herein can confer nuclease protection on LETs under a sigma-70 promoter, we prepared linear templates based on the pBESTOR2—OR1-Pr-UTR1-deGFP-T500 plasmid. 34 Expectedly, overall expression rates were lower compared to the T7 RNAP-based system, and therefore protected linear versus plasmid expression levels were also lower (˜20%, FIG. 4C). Nevertheless, Ter-protected linear templates had a 100-200 fold increase in expression compared to nonprotected LETs (FIG. 4D); making Tus-Ter a suitable platform for rapid prototyping of E. coli RNAP-driven synthetic gene circuits using linear DNA.

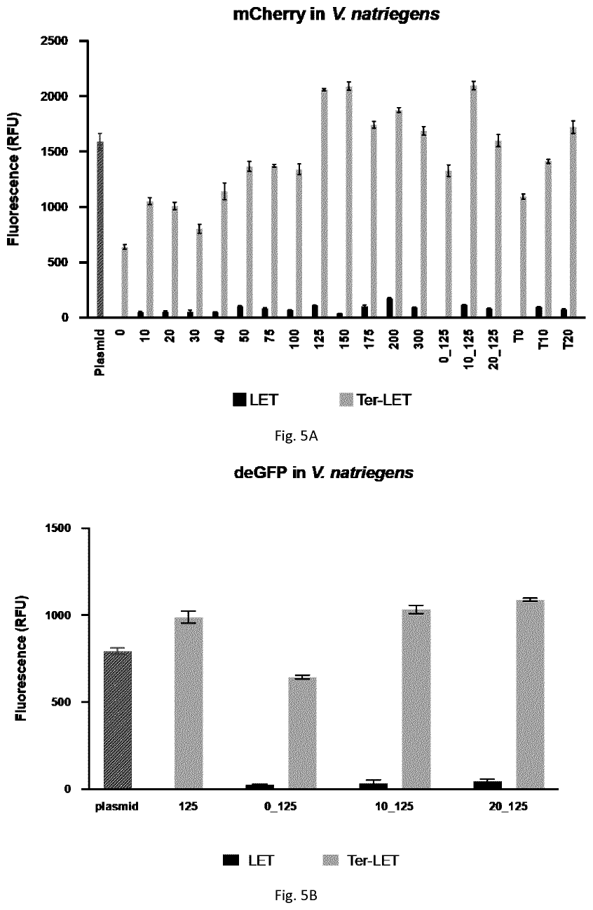

Remarkably, the Tus-Ter constructs and method of this disclosure were also capable of maintaining plasmid-level expression from linear templates in a V. nat CFS. However, here the effects of LET buffer region length as well as T7 terminator were much less pronounced. As seen in FIG. 5, for example, the mCherry LET with only 10 bp buffer on both termini reached 62% of the expression of the LET with 300 bp of buffer (FIG. 5A). For comparison, the same ratio was 10% in E. coli lysate A (FIG. 3A). One possible explanation for this lower dependence on the length of buffer regions is that the exonucleases in V. nat may be less effective against the Tus-Ter complex than the exonucleases in E. coli. Therefore, the temporary dislodgement of Tus on the 3′ terminus by T7 polymerase³³ may not still provide enough opportunity for V. nat exonucleases to fully dismantle Tus-Ter and degrade LETs. Another interesting observation was that, unlike E. coli, in our V. nat lysate mCherry and deGFP both had similar protected-linear vs plasmid expression ratios at 129% and 124%, respectively for corresponding LETs (FIG. 5, see Ter-LET 125). Again, this discrepancy can be due to the divergent regulatory mechanisms in E. coli and V. nat¹⁴. It is worth noting that the overall expression capacity in our V. nat lysate was significantly lower than our E. coli lysates; however, since the scope of this study was demonstrating the utility of Tus-Ter in protection of linear templates, we did not specifically attempt to optimize lysate preparation and reaction conditions to increase overall yield in either lysate. Nonetheless, V. nat CFSs have been previously shown to be capable of reaching E. coli-level productivity¹⁴ and therefore, with the robust LET protection afforded by our Tus-Ter method, there is now an even greater promise in the use of V. nat as the next-generation CFS chassis organism. It should be understood that a terminator is not essential when the constructs of the present disclosure are used to protect DNA sequences that do not transcribe.

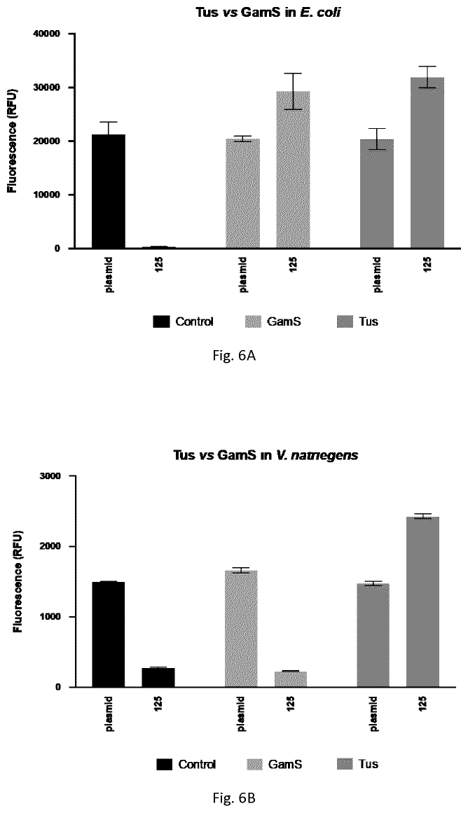

GamS protein has often been used as a gold standard for comparing the LET protection efficiency of new methods in the field^{19,24,25}. In order to see how Tus-Ter compares to GamS, we performed linear vs plasmid expression tests in cell-free reactions containing 5 μM of either Tus or GamS (FIG. 6). In our E. coli lysates, GamS and Tus protected LETs similarly; surpassing plasmid-level expression (FIG. 6A). On the other hand and in accordance with previous reports^{16,19,25}, GamS was completely non-functional in our V. nat lysate; whereas Tus enabled mCherry LETs to reach and surpass plasmid level expression (FIG. 6B). In the process of data collection for this study, two other methods were published wherein they use a similar scheme—terminal blocking of LETs by DNA binding proteins^{19,25}. For one method, termed CroP-LET¹⁹, an 800 bp buffer region at both termini and a one hour preincubation of LETs is required, and the reported equimolar linear vs plasmid protection efficiency is approximately 24% and 2% in E. coli and V. nat extracts, respectively. The other technique 25 uses Ku, a non-specific dsDNA terminus binding protein. However, in the case of Ku reaction conditions aren't described in detail and no linear vs plasmid expression comparison is presented for V. nat extracts, but the ratio is shown to be roughly <10% in E. coli extracts. Therefore, to our knowledge Tus-Ter is the first and only reported LET protection technique that can maintain plasmid-level LET expression in both E. coli and V. nat CFSs.

In terms of implementation, the Tus-Ter system/method presented herein is highly practicable and convenient-requiring minimal manipulations to the cell-free extracts or the linear templates. No strain engineering, or cumbersome post-PCR processing, or prohibitively long buffer regions are required. The Tus protein can be produced and purified from E. coli or other γ-proteobacterial strains in high quantities and added to cell-free reactions immediately before the addition of LETs. Likewise, the 23 bp Ter sequence can be conveniently added during commercial gene synthesis or as a primer overhang during PCR. Our results demonstrate the robust performance of Tus-Ter in two important chassis organisms, the established E. coli and the rapidly emerging V. nat. It has been further demonstrated that Tus-Ter protection of linear DNA can be achieved using endogenously expressed Tus including for example under the control of the endogenous E. coli RNA Polymerase (RNAP). We anticipate that Tus-Ter will be employed widely in research and commercial cell free applications for expedited discovery, especially when V. nat based CFSs are used.

### Example 2

Ter sequences are used as primer extensions in LAMP or RPA for isothermal amplification of low-abundance target pathogen sequences in the presence of Tus. For this, target-specific forward and reverse primers are synthesised with a 5′ Ter overhang. The concomitant binding of amplicons with Tus in the amplification reaction, or even the addition of Tus post-amplification, will result in added stability and therefore sensitivity in diagnostic and gene circuit-based assays; especially when these assays are performed under exonuclease-prone conditions. Such conditions may arise by the use of non-or-partially purified patient samples, or the use of crude enzyme mixtures, or potential residual exonuclease contamination during reaction set up. In all cases terminal blocking of target amplicons is likely to significantly increase their lifetime and thus boost the assays' sensitivity.

### Example 3

Pre-amplification of gene circuit, toehold or aptamer-based biosensing reporter sequences using Ter primers and binding with Tus prior to or during their addition to biological sample, for added stability and sensitivity. Here, toehold reporter or aptamer reporter-specific forward and reverse primers are synthesized with 5′ Ter overhangs and used for PCR amplification of target sequences. As above, if the assay environment is prone to exonuclease contamination such as that from crude enzyme solutions or unpurified biological samples, addition of Tus during or prior to the addition of Ter-reporter sequences can increase reporter stability and therefore assay sensitivity. Further, Tus-Ter can be used as a strand-clamp in gene circuit-based tools where spontaneous breathing or de-hybridization at termini may induce signal leakage, structure de-stabilisation or other failure modes.

### Example 4

Tus is immobilized on the surface of Lateral Flow diagnostic Assays to detect Ter-amplified target pathogen sequences. Here, isothermal amplification (LAMP or RPA) is performed on biological sample using target-specific forward and reverse primers containing functionally oriented Ter and reporter sequence (e.g. aptamer) overhangs. Also, following the general design principles of Lateral Flow Assays (LFAs), Tus is immobilized on two locations (Control and Test) on a standard LFA nitrocellulose strip. Followed by the Tus-Ter immobilization on the Control position of a pre-amplified control reporter construct containing both Ter and an e.g. reporter aptamer. The resulting isothermal amplification reaction solution can then be applied along with a reporter substrate to the Tus LFA strip. If the target pathogen sequence is present in the starting biological sample and successfully amplified, functional, double-stranded Ter and reporter sequences are reconstituted. As a result, the amplicons will be immobilized on the Test position on the LFA strip and the reporter sequences in both Control and test positions will react with the substrate. The user will then be able to detect the signal on each Control and Test position and make a judgement as to the presence or absence of pathogen in the starting biological sample.

### REFERENCES

[1] T. Matsuda, T. Ito, C. Takemoto, K. Katsura, M. Ikeda, M. Wakiyama, M. Kukimoto-Niino, S. Yokoyama, Y. Kurosawa, M. Shirouzu, Cell-free synthesis of functional antibody fragments to provide a structural basis for antibody-antigen interaction, PLOS One. 13 (2018) 1-19. https://doi.org/10.1371/journal.pone.0193158.
[2] P. S. C. Wu, K. Ozawa, S. P. Lim, S. G. Vasudevan, N. E. Dixon, G. Otting, Cell-free transcription/translation from PCR-amplified DNA for high-throughput NMR studies, Angew. Chemie-Int. Ed. 46 (2007) 3356-3358. https://doi.org/10.1002/anie.200605237.
[3] J. Yazaki, M. Galli, A. Y. Kim, K. Nito, F. Aleman, K. N. Chang, A. R. Carvunis, R. Quan, H. Nguyen, L. Song, J. M. Alvarez, S. C. Huang, H. Chen, N. Ramachandran, S. Altmann, R. A. Gutierrez, D. E. Hill, J. I. Schroeder, J. Chory, J. LaBaer, M. Vidal, P. Braun, J. R. Ecker, Mapping transcription factor interactome networks using HaloTag protein arrays, Proc. Natl. Acad. Sci. 113 (2016) E4238-E4247. https://doi.org/10.1073/pnas. 1603229113.
[4] A. S. Karim, M. C. Jewett, A cell-free framework for rapid biosynthetic pathway prototyping and enzyme discovery, Metab. Eng. 36 (2016) 116-126. https://doi.org/10.1016/j.ymben.2016.03.002.
[5] K. A. Woodrow, I. O. Airen, J. R. Swartz, Rapid expression of functional genomic libraries, J. Proteome Res. 5 (2006) 3288-3300. https://doi.org/10.1021/pr050459y.
[6] P. Majkut, I. Claußnitzer, H. Merk, C. Freund, C. P. R. Hackenberger, M. Gerrits, Completion of proteomic data sets by Kd measurement using cell-free synthesis of site-specifically labeled proteins, PLOS One. 8 (2013) 1-11. https://doi.org/10.1371/journal.pone.0082352.
[7] R. Marshall, C. S. Maxwell, S. P. Collins, M. L. Luo, T. Jacobsen, C. L. Beisel, V. Noireaux, Rapid and scalable characterization of CRISPR technologies using an E. coli cell-free transcription-translation system, BioRxiv. (2017) 146-157. https://doi.org/10.1101/169441.
[8] S. S. Yim, N. I. Johns, J. Park, A. L. C. Gomes, R. M. McBee, M. Richardson, C. Ronda, S. P. Chen, D. Garenne, V. Noireaux, H. H. Wang, Multiplex transcriptional characterizations across diverse bacterial species using cell-free systems, Mol. Syst. Biol. 15 (2019) e8875. https://doi.org/https://doi.org/10.15252/msb.20198875.
[9] K. Pardee, A. A. Green, M. K. Takahashi, D. Braff, G. Lambert, J. W. Lee, T. Ferrante, D. Ma, N. Donghia, M. Fan, N. M. Daringer, I. Bosch, D. M. Dudley, D. H. O'Connor, L. Gehrke, J. J. Collins, Rapid, Low-Cost Detection of Zika Virus Using Programmable Biomolecular Components, Cell. 165 (2016) 1255-1266. https://doi.org/10.1016/j.cell.2016.04.059.
[10] J. F. Zawada, G. Yin, A. R. Steiner, J. Yang, A. Naresh, S. M. Roy, D. S. Gold, H. G. Heinsohn, C. J. Murray, Microscale to manufacturing scale-up of cell-free cytokine production-a new approach for shortening protein production development timelines, Biotechnol. Bioeng. 108 (2011) 1570-1578. https://doi.org/10.1002/bit.23103.
[11] A. D. Silverman, A. S. Karim, M. C. Jewett, Cell-free gene expression: an expanded repertoire of applications, Nat. Rev. Genet. 21 (2020) 151-170. https://doi.org/10.1038/s41576-019-0186-3.
[12] J. Hoff, B. Daniel, D. Stukenberg, B. W. Thuronyi, T. Waldminghaus, G. Fritz, Vibrio natriegens: an ultrafast-growing marine bacterium as emerging synthetic biology chassis, Environ. Microbiol. 22 (2020) 4394-4408. https://doi.org/10.1111/1462-2920.15128.
[13] M. T. Weinstock, E. D. Hesek, C. M. Wilson, D. G. Gibson, Vibrio natriegens as a fast-growing host for molecular biology, Nat. Methods. 13 (2016) 849-851. https://doi.org/10.1038/nmeth.3970.
[14] B. J. Des Soye, S. R. Davidson, M. T. Weinstock, D. G. Gibson, M. C. Jewett, Establishing a High-Yielding Cell-Free Protein Synthesis Platform Derived from Vibrio natriegens, ACS Synth. Biol. 7 (2018) 2245-2255. https://doi.org/10.1021/acssynbio.8b00252.
[15] J. Failmezger, S. Scholz, B. Blombach, M. Siemann-Herzberg, Cell-free protein synthesis from fast-growing Vibrio natriegens, Front. Microbiol. 9 (2018) 1-10. https://doi.org/10.3389/fmicb.2018.01146.
[16] D. J. Wiegand, H. H. Lee, N. Ostrov, G. M. Church, Establishing a Cell-Free Vibrio natriegens Expression System, ACS Synth. Biol. 7 (2018) 2475-2479. https://doi.org/10.1021/acssynbio.8b00222.
[17] N. Michel-Reydellet, K. Woodrow, J. Swartz, Increasing PCR fragment stability and protein yields in a cell-free system with genetically modified Escherichia coli extracts, J. Mol. Microbiol. Biotechnol. 9 (2005) 26-34. https://doi.org/10.1159/000088143.
[18] G. R. Smith, How RecBCD Enzyme and Chi Promote DNA Break Repair and Recombination: a Molecular Biologist's View, Microbiol. Mol. Biol. Rev. 76 (2012) 217-228. https://doi.org/10.1128/mmbr.05026-11.
[19] B. Zhu, R. Gan, M. D. Cabezas, T. Kojima, R. Nicol, M. C. Jewett, H. Nakano, Increasing cell-free gene expression yields from linear templates in Escherichia coli and Vibrio natriegens extracts by using DNA-binding proteins, Biotechnol. Bioeng. (2020). https://doi.org/10.1002/bit.27538.
[20] S. M. Schinn, A. Broadbent, W. T. Bradley, B. C. Bundy, Protein synthesis directly from PCR: Progress and applications of cell-free protein synthesis with linear DNA, N. Biotechnol. 33 (2016) 480-487. https://doi.org/10.1016/j.nbt.2016.04.002.
[21] K. Sitaraman, D. Esposito, G. Klarmann, S. F. Le Grice, J. L. Hartley, D. K. Chatterjee, A novel cell-free protein synthesis system, J. Biotechnol. 110 (2004) 257-263. https://doi.org/10.1016/j.jbiotec.2004.02.014.
[22] E. Seki, N. Matsuda, T. Kigawa, Multiple inhibitory factor removal from an Escherichia coli cell extract improves cell-free protein synthesis, J. Biosci. Bioeng. 108 (2009) 30-35. https://doi.org/10.1016/j.jbiosc.2009.02.011.
[23] J. Chappell, K. Jensen, P. S. Freemont, Validation of an entirely in vitro approach for rapid prototyping of DNA regulatory elements for synthetic biology, Nucleic Acids Res. 41 (2013) 3471-3481. https://doi.org/10.1093/nar/gkt052.
[24] R. Marshall, C. S. Maxwell, S. P. Collins, C. L. Beisel, V. Noireaux, Short DNA containing χ sites enhances DNA stability and gene expression in E. coli cell-free transcription-translation systems, Biotechnol. Bioeng. 114 (2017) 2137-2141. https://doi.org/10.1002/bit.26333.
[25] S. S. Yim, N. I. Johns, V. Noireaux, H. H. Wang, Protecting linear DNA templates in cell-free expression systems from diverse bacteria, ACS Synth. Biol. (2020) 0-4. https://doi.org/10.1021/acssynbio.0c00277.
[26] M. Wilkinson, L. Troman, W. A. Wan Nur Ismah, Y. Chaban, M. B. Avison, M. S. Dillingham, D. B. Wigley, Structural basis for the inhibition of RecBCD by Gam and its synergistic antibacterial effect with quinolones, Elife. 5 (2016) 1-12. https://doi.org/10.7554/elife.22963.
[27] B. A. Berghuis, V. S. Raducanu, M. M. Elshenawy, S. Jergic, M. Depken, N. E. Dixon, S. M. Hamdan, N. H. Dekker, What is all this fuss about Tus? Comparison of recent findings from biophysical and biochemical experiments, Crit. Rev. Biochem. Mol. Biol. 53 (2018) 49-63. https://doi.org/10.1080/10409238.2017.1394264.
[28] E. Galli, J. L. Ferat, J. M. Desfontaines, M. E. Val, O. Skovgaard, F. X. Barre, C. Possoz, Replication termination without a replication fork trap, Sci. Rep. 9 (2019) 1-11. https://doi.org/10.1038/s41598-019-43795-2.
[29] P. A. Gottlieb, S. Wu, X. Zhang, M. Tecklenburg, P. Kuempel, T. M. Hill, Equilibrium, kinetic, and footprinting studies of the Tus-Ter protein-DNA interaction, J. Biol. Chem. 267 (1992) 7434-7443.
[30] E. H. Lee, A. Kornberg, M. Hidaka, T. Kobayashi, T. Horiuchi, Escherichia coli replication termination protein impedes the action of helicases, Proc. Natl. Acad. Sci. U. 86 (1989) 9104-9108. S. A. https://doi.org/10.1073/pnas.86.23.9104.
[31] M. Pandey, M. M. Elshenawy, S. Jergic, M. Takahashi, N. E. Dixon, S. M. Hamdan, S. S. Patel, Two mechanisms coordinate replication termination by the Escherichia coli Tus-Ter complex, Nucleic Acids Res. 43 (2015) 5924-5935. https://doi.org/10.1093/nar/gkv527.
[32] M. Z. Levine, N. E. Gregorio, M. C. Jewett, K. R. Watts, J. P. Oza, Escherichia coli-Based Cell-Free Protein Synthesis: Protocols for a robust, flexible, and accessible platform technology, J. Vis. Exp. (2019) 1-11. https://doi.org/10.3791/58882.
[33] F. Caschera, V. Noireaux, Preparation of amino acid mixtures for cell-free expression systems, Biotechniques. 58 (2015) 40-43. https://doi.org/10.2144/000114249.
[34] R. Guajardo, R. Sousa, Characterization of the effects of Escherichia coli replication terminator protein (Tus) on transcription reveals dynamic nature of the Tus block to transcription complex progression, Nucleic Acids Res. 27 (1999) 2814-2824. https://doi.org/10.1093/nar/27.13.2814.
[35] J. H. Ahn, H. S. Chu, T. W. Kim, I. S. Oh, C. Y. Choi, G. H. Hahn, C. G. Park, D. M. Kim, Cell-free synthesis of recombinant proteins from PCR-amplified genes at a comparable productivity to that of plasmid-based reactions, Biochem. Biophys. Res. Commun. 338 (2005) 1346-1352. https://doi.org/10.1016/j.bbrc.2005.10.094.
[30] Z. Z. Sun, E. Yeung, C. A. Hayes, V. Noireaux, R. M. Murray, Linear DNA for rapid prototyping of synthetic biological circuits in an Escherichia coli based TX-TL cell-free system, ACS Synth. Biol. 3 (2014) 387-397. https://doi.org/10.1021/sb400131a.

TABLE 1
Sequence Listings
Coding Sequences

| | SEQ | |
|---|---|---|
| | ID | |
| Gene | NO | Sequence |
| x6His- | 7 | atgcatcaccatcaccatcacaacaaagaaattttggctgtagttgaagccgtatccaatgaaaaggcgct |
| NusA-Tev- | | acctcgcgagaagattttcgaagcattggaaagcgcgctggcgacagcaacaaagaaaaaatatgaac |
| Tus | | aagagatcgacgtccgcgtacagatcgatcgcaaaagcggtgattttgacactttccgtcgctggttagtt |
| | | gttgatgaagtcacccagccgaccaaggaaatcacccttgaagccgcacgttatgaagatgaaagcctg |
| | | aacctgggcgattacgttgaagatcagattgagtctgttacctttgaccgtatcactacccagacggcaaa |
| | | acaggttatcgtgcagaaagtgcgtgaagccgaacgtgcgatggtggttgatcagttccgtgaacacga |
| | | aggtgaaatcatcaccggcgtggtgaaaaaagtaaaccgcgacaacatctctctggatctgggcaacaa |
| | | cgctgaagccgtgatcctgcgcgaagatatgctgccgcgtgaaaacttccgccctggcgaccgcgttcg |
| | | tggcgtgctctattccgttcgcccggaagcgcgtggcgcgcaactgttcgtcactcgttccaagccggaa |
| | | atgctgatcgaactgttccgtattgaagtgccagaaatcggcgaagaagtgattgaaattaaagcagcgg |
| | | ctcgcgatccgggttctcgtgcgaaaatcgcggtgaaaaccaacgataaacgtatcgatccggtaggtg |
| | | cttgcgtaggtatgcgtggcgcgcgtgttcaggcggtgtctactgaactgggtggcgagcgtatcgatat |
| | | cgtcctgtgggatgataacccggcgcagttcgtgattaacgcaatggcaccggcagacgttgcttctatc |
| | | gtggtggatgaagataaacacaccatggatatcgccgttgaagccggtaacctggcgcaggcgattgg |
| | | ccgtaacggtcagaacgtgcgtctggcttcgcagctgagcggttgggaactcaacgtgatgaccgttga |
| | | cgacctgcaggctaagcatcaggcggaagcgcacgcagcgatcgacaccttcaccaaatatctcgaca |
| | | tcgacgaagacttcgcgactgttctggtagaagaaggcttctcgacgctggaagaattggcctatgtgcc |
| | | gatgaaagagctgttggaaatcgaaggccttgatgagccgaccgttgaagcactgcgcgagcgtgcta |
| | | aaaatgcactggccaccattgcacaggcccaggaagaaagcctcggtgataacaaaccggctgacgat |
| | | ctgctgaaccttgaaggggtagatcgtgatttggcattcaaactggccgcccgtggcgtttgtacgctgga |
| | | agatctcgccgaacagggcattgatgatctggctgatatcgaagggttgaccgacgaaaaagccggag |
| | | cactgattatggctgcccgtaacatttgctggttcggtgacgaagcggattacgacatcccaacgaccga |
| | | aaacctgtattttcagggcatggcgcgttacgatctcgtagaccgactcaacactacctttcgccagatgg |
| | | aacaagagctggctatatttgccgctcatcttgagcaacacaagctattggttgcccgcgtgttctctttgcc |
| | | ggaggtaaaaaaagaggatgagcataatccgcttaatcgtattgaggtaaaacaacatctcggcaacga |
| | | cgcgcagtcgctggcgttgcgtcatttccgccatttatttattcaacaacagtccgaaaatcgcagcagca |
| | | aggccgctgtccgtctgcctggcgtgttgtgttaccaggtcgataacctttcgcaagcagcgttggtcagt |
| | | catattcagcacatcaataaactcaagaccacgttcgagcatatcgtcacggttgaatcagaactccccac |
| | | cgcggcacgttttgaatgggtgcatcgtcatttgccggggctgatcacccttaatgcttaccgcacgctca |
| | | ccgttctgcacgaccccgccactttacgctttggttgggctaataaacatatcattaagaatttacatcgtga |
| | | tgaagtcctggcacagctggaaaaaagcctgaaatcaccacgcagtgtcgcaccgtggacgcgcgag |
| | | gagtggcaaagaaaactggagcgagagtatcaggatatcgctgccctgccacagaacgcgaagttaaa |
| | | aatcaaacgtccggtgaaggtgcagccgattgcccgcgtctggtacaaaggagatcaaaaacaagtcc |
| | | aacacgcctgccctacaccactgattgcactgattaatcgggataatggcgcgggcgtgccggacgttg |
| | | gtgagttgttaaattacgatgccgacaatgtgcagcaccgttataaacctcaggcgcagccgcttcgtttg |
| | | atcattccacggctgcacctgtatgttgcagattaa |
| mCherry | 8 | atggtctcaaagggggaagaggataatatggcgatcatcaaggagtttatgcgtttcaaagttcatatgga |
| | | aggcagtgttaacggccacgagttcgagattgagggggagggggaaggacgcccttacgaagggac |
| | | ccaaacagctaaattaaaagtgactaaagggggtccgttgcctttcgcgtgggatatcttatcaccgcagt |
| | | tcatgtatggatcgaaggcttatgtaaaacaccccgcagacatccctgattaccttaaactttcattcccgg |
| | | aagggtttaaatgggagcgtgtaatgaattttgaggacggaggggtagttacggtcactcaagatagcag |
| | | tttacaagatggagagttcatttataaggtgaagttacgtggaacaaacttcccgtcagacggtcccgttat |
| | | gcaaaaaaagacgatgggctgggaggcttcttccgagcgcatgtatccagaagacggtgcactgaaag |
| | | gcgagatcaagcaacgcctgaaattaaaggacgggggacattatgacgccgaggtcaaaactacatac |
| | | aaagctaagaaacccgttcagttaccaggtgcctacaacgtaaacattaaattggacattacgtcgcataa |
| | | cgaggattacacgatcgtggaacaatatgaacgtgctgagggtcgccactccaccgggggtatggacg |
| | | agctttataaataa |
| deGFP | 9 | atggagcttttcactggcgttgttcccatcctggtcgagctggacggcgacgtaaacggccacaagttca |
| | | gcgtgtccggcgagggcgagggcgatgccacctacggcaagctgaccctgaagttcatctgcaccac |
| | | cggcaagctgcccgtgccctggcccaccctcgtgaccaccctgacctacggcgtgcagtgcttcagcc |
| | | gctaccccgaccacatgaagcagcacgacttcttcaagtccgccatgcccgaaggctacgtccaggag |
| | | cgcaccatcttcttcaaggacgacggcaactacaagacccgcgccgaggtgaagttcgagggcgaca |
| | | ccctggtgaaccgcatcgagctgaagggcatcgacttcaaggaggacggcaacatcctggggcacaa |
| | | gctggagtacaactacaacagccacaacgtctatatcatggccgacaagcagaagaacggcatcaagg |
| | | tgaacttcaagatccgccacaacatcgaggacggcagcgtgcagctcgccgaccactaccagcagaa |
| | | cacccccatcggcgacggccccgtgctgctgcccgacaaccactacctgagcacccagtccgccctg |
| | | agcaaagaccccaacgagaagcgcgatcacatggtcctgctggagttcgtgaccgccgccgggatcta |
| | | a |
| x6His-Tev- | 10 | atgcatcaccatcaccatcacgattacgacatcccaacgaccgaaaacctgtattttcagggcatgaaca |
| T7 RNA | | cgattaacatcgctaagaacgacttctctgacatcgaactggctgctatcccgttcaacactctggctgac |
| Polymerase | | cattacggtgagcgtttagctcgcgaacagttggcccttgagcatgagtcttacgagatgggtgaagcac |
| | | gcttccgcaagatgtttgagcgtcaacttaaagctggtgaggttgcggataacgctgccgccaagcctct |
| | | catcactaccctactccctaagatgattgcacgcatcaacgactggtttgaggaagtgaaagctaagcgc |
| | | ggcaagcgcccgacagccttccagttcctgcaagaaatcaagccggaagccgtagcgtacatcaccat |
| | | taagaccactctggcttgcctaaccagtgctgacaatacaaccgttcaggctgtagcaagcgcaatcggt |
| | | cgggccattgaggacgaggctcgcttcggtcgtatccgtgaccttgaagctaagcacttcaagaaaaac |
| | | gttgaggaacaactcaacaagcgcgtagggcacgtctacaagaaagcatttatgcaagttgtcgaggct |
| | | gacatgctctctaagggtctactcggtggcgaggcgtggtcttcgtggcataaggaagactctattcatgt |
| | | aggagtacgctgcatcgagatgctcattgagtcaaccggaatggttagcttacaccgccaaaatgctggc |
| | | gtagtaggtcaagactctgagactategaactcgcacctgaatacgctgaggctatcgcaacccgtgca |
| | | ggtgcgctggctggcatctctccgatgttccaaccttgcgtagttcctcctaagccgtggactggcattact |
| | | ggtggtggctattgggctaacggtcgtcgtcctctggcgctggtgcgtactcacagtaagaaagcactga |
| | | tgcgctacgaagacgtttacatgcctgaggtgtacaaagcgattaacattgcgcaaaacaccgcatgga |
| | | aaatcaacaagaaagtcctagcggtcgccaacgtaatcaccaagtggaagcattgtccggtcgaggac |
| | | atccctgcgattgagcgtgaagaactcccgatgaaaccggaagacatcgacatgaatcctgaggctctc |
| | | accgcgtggaaacgtgctgccgctgctgtgtaccgcaaggacaaggctcgcaagtctcgccgtatcag |
| | | ccttgagttcatgcttgagcaagccaataagtttgctaaccataaggccatctggttcccttacaacatgga |
| | | ctggcgcggtcgtgtttacgctgtgtcaatgttcaacccgcaaggtaacgatatgaccaaaggactgctta |
| | | cgctggcgaaaggtaaaccaatcggtaaggaaggttactactggctgaaaatccacggtgcaaactgtg |
| | | cgggtgtcgataaggttccgttccctgagcgcatcaagttcattgaggaaaaccacgagaacatcatggc |
| | | ttgcgctaagtctccactggagaacacttggtgggctgagcaagattctccgttctgcttccttgogttctgc |
| | | tttgagtacgctggggtacagcaccacggcctgagctataactgctcccttccgctggcgtttgacgggt |
| | | cttgctctggcatccagcacttctccgcgatgctccgagatgaggtaggtggtcgcgcggttaacttgctt |
| | | cctagtgaaaccgttcaggacatctacgggattgttgctaagaaagtcaacgagattctacaagcagacg |
| | | caatcaatgggaccgataacgaagtagttaccgtgaccgatgagaacactggtgaaatctctgagaaag |
| | | tcaagctgggcactaaggcactggctggtcaatggctggcttacggtgttactcgcagtgtgactaagcg |
| | | ttcagtcatgacgctggcttacgggtccaaagagttcggcttccgtcaacaagtgctggaagataccattc |
| | | agccagctattgattccggcaagggtctgatgttcactcagccgaatcaggctgctggatacatggctaa |
| | | gctgatttgggaatctgtgagcgtgacggtggtagctgcggttgaagcaatgaactggcttaagtctgctg |
| | | ctaagctgctggctgctgaggtcaaagataagaagactggagagattcttcgcaagcgttgcgctgtgca |
| | | ttgggtaactcctgatggtttccctgtgtggcaggaatacaagaagcctattcagacgcgcttgaacctga |
| | | tgttcctcggtcagttccgcttacagcctaccattaacaccaacaaagatagcgagattgatgcacacaaa |
| | | caggagtctggtatcgctcctaactttgtacacagccaagacggtagccaccttcgtaagactgtagtgtg |
| | | ggcacacgagaagtacggaatcgaatcttttgcactgattcacgactccttcggtaccattccggctgacg |
| | | ctgcgaacctgttcaaagcagtgcgcgaaactatggttgacacatatgagtcttgtgatgtactggctgatt |
| | | tctacgaccagttcgctgaccagttgcacgagtctcaattggacaaaatgccagcacttccggctaaagg |
| | | taacttgaacctccgtgacatcttagagtcggacttcgcgttcgcgtaa |

LET Sequences

| LET | | |
|---|---|---|
| Name | mCherry | Ter-mCherry |
| 0 | taatacgactcactatagggagaccacaacggtttccctct | ggctccgaataagtatgttgtaactaaagtgtaatacgactc |
| | agaaataattttgtttaactttaagaaggagatatacatatgg | actatagggagaccacaacggtttccctctagaaataatttt |
| | tctcaaagggggaagaggataatatggcgatcatcaagg | gtttaactttaagaaggagatatacatatggtctcaaaggg |
| | agtttatgcgtttcaaagttcatatggaaggcagtgttaacg | ggaagaggataatatggcgatcatcaaggagtttatgcgtt |
| | gccacgagttcgagattgagggggagggggaaggacg | tcaaagttcatatggaaggcagtgttaacggccacgagttc |
| | cccttacgaagggacccaaacagctaaattaaaagtgact | gagattgagggggagggggaaggacgcccttacgaag |
| | aaagggggtccgttgcctttcgcgtgggatatcttatcacc | ggacccaaacagctaaattaaaagtgactaaagggggtc |
| | gcagttcatgtatggatcgaaggcttatgtaaaacaccccg | cgttgcctttcgcgtgggatatcttatcaccgcagttcatgt |
| | cagacatccctgattaccttaaactttcattcccggaagggt | atggatcgaaggcttatgtaaaacaccccgcagacatccct |
| | ttaaatgggagcgtgtaatgaattttgaggacggaggggt | gattaccttaaactttcattcccggaagggtttaaatgggag |
| | agttacggtcactcaagatagcagtttacaagatggagagt | cgtgtaatgaattttgaggacggaggggtagttacggtca |
| | tcatttataaggtgaagttacgtggaacaaacttcccgtcag | ctcaagatagcagtttacaagatggagagttcatttataag |
| | acggtcccgttatgcaaaaaaagacgatgggctgggagg | gtgaagttacgtggaacaaacttcccgtcagacggtcccg |
| | cttcttccgagcgcatgtatccagaagacggtgcactgaa | ttatgcaaaaaaagacgatgggctgggaggcttcttccga |
| | aggcgagatcaagcaacgcctgaaattaaaggacgggg | gcgcatgtatccagaagacggtgcactgaaaggcgagat |
| | gacattatgacgccgaggtcaaaactacatacaaagctaa | caagcaacgcctgaaattaaaggacgggggacattatga |
| | gaaacccgttcagttaccaggtgcctacaacgtaaacatta | cgccgaggtcaaaactacatacaaagctaagaaacccgtt |
| | aattggacattacgtcgcataacgaggattacacgatcgtg | cagttaccaggtgcctacaacgtaaacattaaattggacat |
| | gaacaatatgaacgtgctgagggtcgccactccaccggg | tacgtcgcataacgaggattacacgatcgtggaacaatat |
| | ggtatggacgagctttataaataa (SEQ ID NO: 11) | gaacgtgctgagggtcgccactccaccgggggtatggac |
| | | gagctttataaataacactttagttacaacatacttattgcc |
| | | tcgg (SEQ ID NO: 12) |
| 10 | cccgcgaaattaatacgactcactatagggagaccacaac | ggctccgaataagtatgttgtaactaaagtgcccgcgaaat |
| | ggtttccctctagaaataattttgtttaactttaagaaggaga | taatacgactcactatagggagaccacaacggtttccctct |
| | tatacatatggtctcaaagggggaagaggataatatggcga | agaaataattttgtttaactttaagaaggagatatacatatgg |
| | tcatcaaggagtttatgcgtttcaaagttcatatggaaggca | tctcaaagggggaagaggataatatggcgatcatcaagg |
| | gtgttaacggccacgagttcgagattgagggggagggg | agtttatgcgtttcaaagttcatatggaaggcagtgttaacg |
| | gaaggacgcccttacgaagggacccaaacagctaaatta | gccacgagttcgagattgagggggagggggaaggacg |
| | aaagtgactaaagggggtccgttgcctttcgcgtgggatat | cccttacgaagggacccaaacagctaaattaaaagtgact |
| | cttatcaccgcagttcatgtatggatcgaaggcttatgtaaa | aaagggggtccgttgcctttcgcgtgggatatcttatcacc |
| | acaccccgcagacatccctgattaccttaaactttcattccc | gcagttcatgtatggatcgaaggcttatgtaaaacaccccg |
| | ggaagggtttaaatgggagcgtgtaatgaattttgaggac | cagacatccctgattaccttaaactttcattcccggaagggt |
| | ggaggggtagttacggtcactcaagatagcagtttacaag | ttaaatgggagcgtgtaatgaattttgaggacggaggggt |
| | atggagagttcatttataaggtgaagttacgtggaacaaac | agttacggtcactcaagatagcagtttacaagatggagagt |
| | ttcccgtcagacggtcccgttatgcaaaaaaagacgatgg | tcatttataaggtgaagttacgtggaacaaacttcccgtcag |
| | gctgggaggcttcttccgagcgcatgtatccagaagacg | acggtcccgttatgcaaaaaaagacgatgggctgggagg |
| | gtgcactgaaaggcgagatcaagcaacgcctgaaattaa | cttcttccgagcgcatgtatccagaagacggtgcactgaa |
| | aggacgggggacattatgacgccgaggtcaaaactacat | aggcgagatcaagcaacgcctgaaattaaaggacgggg |
| | acaaagctaagaaacccgttcagttaccaggtgcctacaa | gacattatgacgccgaggtcaaaactacatacaaagctaa |
| | cgtaaacattaaattggacattacgtcgcataacgaggatt | gaaacccgttcagttaccaggtgcctacaacgtaaacatta |
| | acacgatcgtggaacaatatgaacgtgctgagggtcgcc | aattggacattacgtcgcataacgaggattacacgatcgtg |
| | actccaccgggggtatggacgagctttataaataatgagat | gaacaatatgaacgtgctgagggtcgccactccaccggg |
| | ccgg (SEQ ID NO: 13) | ggtatggacgagctttataaataatgagatccggcacttta |
| | | gttacaacatacttattgcctcgg (SEQ ID NO: 14) |
| 20 | agatctcgatcccgcgaaattaatacgactcactataggga | ggctccgaataagtatgttgtaactaaagtgagatctcgat |
| | gaccacaacggtttccctctagaaataattttgtttaacttta | cccgcgaaattaatacgactcactatagggagaccacaac |
| | agaaggagatatacatatggtctcaaagggggaagaggat | ggtttccctctagaaataattttgtttaactttaagaaggaga |
| | aatatggcgatcatcaaggagtttatgcgtttcaaagttcat | tatacatatggtctcaaagggggaagaggataatatggcga |
| | atggaaggcagtgttaacggccacgagttcgagattgag | tcatcaaggagtttatgcgtttcaaagttcatatggaaggca |
| | ggggagggggaaggacgcccttacgaagggacccaaa | gtgttaacggccacgagttcgagattgagggggagggg |
| | cagctaaattaaaagtgactaaagggggtccgttgcctttc | gaaggacgcccttacgaagggacccaaacagctaaatta |
| | gcgtgggatatcttatcaccgcagttcatgtatggatcgaa | aaagtgactaaagggggtccgttgcctttcgcgtgggatat |
| | ggcttatgtaaaacaccccgcagacatccctgattacctta | cttatcaccgcagttcatgtatggatcgaaggcttatgtaaa |
| | aactttcattcccggaagggtttaaatgggagcgtgtaatg | acaccccgcagacatccctgattaccttaaactttcattccc |
| | aattttgaggacggaggggtagttacggtcactcaagata | ggaagggtttaaatgggagcgtgtaatgaattttgaggac |
| | gcagtttacaagatggagagttcatttataaggtgaagttac | ggaggggtagttacggtcactcaagatagcagtttacaag |
| | gtggaacaaacttcccgtcagacggtcccgttatgcaaaa | atggagagttcatttataaggtgaagttacgtggaacaaac |
| | aaagacgatgggctgggaggcttcttccgagcgcatgtat | ttcccgtcagacggtcccgttatgcaaaaaaagacgatgg |
| | ccagaagacggtgcactgaaaggcgagatcaagcaacg | gctgggaggcttcttccgagcgcatgtatccagaagacg |
| | cctgaaattaaaggacgggggacattatgacgccgaggt | gtgcactgaaaggcgagatcaagcaacgcctgaaattaa |
| | caaaactacatacaaagctaagaaacccgttcagttacca | aggacgggggacattatgacgccgaggtcaaaactacat |
| | ggtgcctacaacgtaaacattaaattggacattacgtcgca | acaaagctaagaaacccgttcagttaccaggtgcctacaa |
| | taacgaggattacacgatcgtggaacaatatgaacgtgct | cgtaaacattaaattggacattacgtcgcataacgaggatt |
| | gagggtcgccactccaccgggggtatggacgagctttat | acacgatcgtggaacaatatgaacgtgctgagggtcgcc |
| | aaataatgagatccggctgctaacaa (SEQ ID NO: | actccaccgggggtatggacgagctttataaataatgagat |
| | 15) | ccggctgctaacaacactttagttacaacatacttattgcct |
| | | cgg (SEQ ID NO: 16) |
| 30 | tagaggatcgagatctcgatcccgcgaaattaatacgact | ggctccgaataagtatgttgtaactaaagtgtagaggatcg |
| | cactatagggagaccacaacggtttccctctagaaataatt | agatctcgatcccgcgaaattaatacgactcactataggga |
| | ttgtttaactttaagaaggagatatacatatggtctcaaagg | gaccacaacggtttccctctagaaataattttgtttaacttta |
| | gggaagaggataatatggcgatcatcaaggagtttatgcg | agaaggagatatacatatggtctcaaagggggaagaggat |
| | tttcaaagttcatatggaaggcagtgttaacggccacgagt | aatatggcgatcatcaaggagtttatgcgtttcaaagttcat |
| | tcgagattgagggggagggggaaggacgcccttacgaa | atggaaggcagtgttaacggccacgagttcgagattgag |
| | gggacccaaacagctaaattaaaagtgactaaagggggt | ggggagggggaaggacgcccttacgaagggacccaaa |
| | ccgttgcctttcgcgtgggatatcttatcaccgcagttcatgt | cagctaaattaaaagtgactaaagggggtccgttgcctttc |
| | atggatcgaaggcttatgtaaaacaccccgcagacatccc | gcgtgggatatcttatcaccgcagttcatgtatggatcgaa |
| | tgattaccttaaactttcattcccggaagggtttaaatggga | ggcttatgtaaaacaccccgcagacatccctgattacctta |
| | gcgtgtaatgaattttgaggacggaggggtagttacggtc | aactttcattcccggaagggtttaaatgggagcgtgtaatg |
| | actcaagatagcagtttacaagatggagagttcatttataag | aattttgaggacggaggggtagttacggtcactcaagata |
| | gtgaagttacgtggaacaaacttcccgtcagacggtcccg | gcagtttacaagatggagagttcatttataaggtgaagttac |
| | ttatgcaaaaaaagacgatgggctgggaggcttcttccga | gtggaacaaacttcccgtcagacggtcccgttatgcaaaa |
| | gcgcatgtatccagaagacggtgcactgaaaggcgagat | aaagacgatgggctgggaggcttcttccgagcgcatgtat |
| | caagcaacgcctgaaattaaaggacgggggacattatga | ccagaagacggtgcactgaaaggcgagatcaagcaacg |
| | cgccgaggtcaaaactacatacaaagctaagaaacccgtt | cctgaaattaaaggacgggggacattatgacgccgaggt |
| | cagttaccaggtgcctacaacgtaaacattaaattggacat | caaaactacatacaaagctaagaaacccgttcagttacca |
| | tacgtcgcataacgaggattacacgatcgtggaacaatat | ggtgcctacaacgtaaacattaaattggacattacgtcgca |
| | gaacgtgctgagggtcgccactccaccgggggtatggac | taacgaggattacacgatcgtggaacaatatgaacgtgct |
| | gagctttataaataatgagatccggctgctaacaaagccc | gagggtcgccactccaccgggggtatggacgagctttat |
| | gaaag (SEQ ID NO: 17) | aaataatgagatccggctgctaacaaagcccgaaagcac |
| | | tttagttacaacatacttattgcctcgg (SEQ ID NO: |
| | | 18) |
| 40 | gcgtccggcgtagaggatcgagatctcgatcccgcgaaa | ggctccgaataagtatgttgtaactaaagtggcgtccggc |
| | ttaatacgactcactatagggagaccacaacggtttccctc | gtagaggatcgagatctcgatcccgcgaaattaatacgac |
| | tagaaataattttgtttaactttaagaaggagatatacatatg | tcactatagggagaccacaacggtttccctctagaaataat |
| | gtctcaaagggggaagaggataatatggcgatcatcaag | tttgtttaactttaagaaggagatatacatatggtctcaaagg |
| | gagtttatgcgtttcaaagttcatatggaaggcagtgttaac | gggaagaggataatatggcgatcatcaaggagtttatgcg |
| | ggccacgagttcgagattgagggggagggggaaggac | tttcaaagttcatatggaaggcagtgttaacggccacgagt |
| | gcccttacgaagggacccaaacagctaaattaaaagtga | tcgagattgagggggagggggaaggacgcccttacgaa |
| | ctaaagggggtccgttgcctttcgcgtgggatatcttatcac | gggacccaaacagctaaattaaaagtgactaaagggggt |
| | cgcagttcatgtatggatcgaaggcttatgtaaaacacccc | ccgttgcctttcgcgtgggatatcttatcaccgcagttcatgt |
| | gcagacatccctgattaccttaaactttcattcccggaagg | atggatcgaaggcttatgtaaaacaccccgcagacatccc |
| | gtttaaatgggagcgtgtaatgaattttgaggacggaggg | tgattaccttaaactttcattcccggaagggtttaaatggga |
| | gtagttacggtcactcaagatagcagtttacaagatggaga | gcgtgtaatgaattttgaggacggaggggtagttacggtc |
| | gttcatttataaggtgaagttacgtggaacaaacttcccgtc | actcaagatagcagtttacaagatggagagttcatttataag |
| | agacggtcccgttatgcaaaaaaagacgatgggctggga | gtgaagttacgtggaacaaacttcccgtcagacggtcccg |
| | ggcttcttccgagcgcatgtatccagaagacggtgcactg | ttatgcaaaaaaagacgatgggctgggaggcttcttccga |
| | aaaggcgagatcaagcaacgcctgaaattaaaggacgg | gcgcatgtatccagaagacggtgcactgaaaggcgagat |
| | gggacattatgacgccgaggtcaaaactacatacaaagct | caagcaacgcctgaaattaaaggacgggggacattatga |
| | aagaaacccgttcagttaccaggtgcctacaacgtaaaca | cgccgaggtcaaaactacatacaaagctaagaaacccgtt |
| | ttaaattggacattacgtcgcataacgaggattacacgatc | cagttaccaggtgcctacaacgtaaacattaaattggacat |
| | gtggaacaatatgaacgtgctgagggtcgccactccacc | tacgtcgcataacgaggattacacgatcgtggaacaatat |
| | gggggtatggacgagctttataaataatgagatccggctg | gaacgtgctgagggtcgccactccaccgggggtatggac |
| | ctaacaaagcccgaaaggaagctgagt (SEQ ID | gagctttataaataatgagatccggctgctaacaaagccc |
| | NO: 19) | gaaaggaagctgagtcactttagttacaacatacttattgcc |
| | | tcgg (SEQ ID NO: 20) |
| 50 | cggccacgatgcgtccggcgtagaggatcgagatctcga | ggctccgaataagtatgttgtaactaaagtgcggccacgat |
| | tcccgcgaaattaatacgactcactatagggagaccacaa | gcgtccggcgtagaggatcgagatctcgatcccgcgaaa |
| | cggtttccctctagaaataattttgtttaactttaagaaggag | ttaatacgactcactatagggagaccacaacggtttccctc |
| | atatacatatggtctcaaagggggaagaggataatatggc | tagaaataattttgtttaactttaagaaggagatatacatatg |
| | gatcatcaaggagtttatgcgtttcaaagttcatatggaagg | gtctcaaagggggaagaggataatatggcgatcatcaag |
| | cagtgttaacggccacgagttcgagattgagggggaggg | gagtttatgcgtttcaaagttcatatggaaggcagtgttaac |
| | ggaaggacgcccttacgaagggacccaaacagctaaatt | ggccacgagttcgagattgagggggagggggaaggac |
| | aaaagtgactaaagggggtccgttgcctttcgcgtgggat | gcccttacgaagggacccaaacagctaaattaaaagtga |
| | atcttatcaccgcagttcatgtatggatcgaaggcttatgta | ctaaagggggtccgttgcctttcgcgtgggatatcttatcac |
| | aaacaccccgcagacatccctgattaccttaaactttcattc | cgcagttcatgtatggatcgaaggcttatgtaaaacacccc |
| | ccggaagggtttaaatgggagcgtgtaatgaattttgagg | gcagacatccctgattaccttaaactttcattcccggaagg |
| | acggaggggtagttacggtcactcaagatagcagtttaca | gtttaaatgggagcgtgtaatgaattttgaggacggaggg |
| | agatggagagttcatttataaggtgaagttacgtggaacaa | gtagttacggtcactcaagatagcagtttacaagatggaga |
| | acttcccgtcagacggtcccgttatgcaaaaaaagacgat | gttcatttataaggtgaagttacgtggaacaaacttcccgtc |
| | gggctgggaggcttcttccgagcgcatgtatccagaaga | agacggtcccgttatgcaaaaaaagacgatgggctggga |
| | cggtgcactgaaaggcgagatcaagcaacgcctgaaatt | ggcttcttccgagcgcatgtatccagaagacggtgcactg |
| | aaaggacgggggacattatgacgccgaggtcaaaactac | aaaggcgagatcaagcaacgcctgaaattaaaggacgg |
| | atacaaagctaagaaacccgttcagttaccaggtgcctac | gggacattatgacgccgaggtcaaaactacatacaaagct |
| | aacgtaaacattaaattggacattacgtcgcataacgagga | aagaaacccgttcagttaccaggtgcctacaacgtaaaca |
| | ttacacgatcgtggaacaatatgaacgtgctgagggtcgc | ttaaattggacattacgtcgcataacgaggattacacgatc |
| | cactccaccgggggtatggacgagctttataaataatgag | gtggaacaatatgaacgtgctgagggtcgccactccacc |
| | atccggctgctaacaaagcccgaaaggaagctgagttgg | gggggtatggacgagctttataaataatgagatccggctg |
| | ctgctgc (SEQ ID NO: 21) | ctaacaaagcccgaaaggaagctgagttggctgctgcca |
| | | ctttagttacaacatacttattgcctcgg (SEQ ID NO: |
| | | 22) |
| 75 | accgcacctgtggcgccggtgatgccggccacgatgcgt | ggctccgaataagtatgttgtaactaaagtgaccgcacctg |
| | ccggcgtagaggatcgagatctcgatcccgcgaaattaat | tggcgccggtgatgccggccacgatgcgtccggcgtag |
| | acgactcactatagggagaccacaacggtttccctctaga | aggatcgagatctcgatcccgcgaaattaatacgactcact |
| | aataattttgtttaactttaagaaggagatatacatatggtct | atagggagaccacaacggtttccctctagaaataattttgtt |
| | caaagggggaagaggataatatggcgatcatcaaggagtt | taactttaagaaggagatatacatatggtctcaaaggggga |
| | tatgcgtttcaaagttcatatggaaggcagtgttaacggcc | agaggataatatggcgatcatcaaggagtttatgcgtttca |
| | acgagttcgagattgagggggagggggaaggacgccct | aagttcatatggaaggcagtgttaacggccacgagttcga |
| | tacgaagggacccaaacagctaaattaaaagtgactaaa | gattgagggggagggggaaggacgcccttacgaaggg |
| | gggggtccgttgcctttcgcgtgggatatcttatcaccgca | acccaaacagctaaattaaaagtgactaaagggggtccgt |
| | gttcatgtatggatcgaaggcttatgtaaaacaccccgcag | tgcctttcgcgtgggatatcttatcaccgcagttcatgtatg |
| | acatccctgattaccttaaactttcattcccggaagggtttaa | gatcgaaggcttatgtaaaacaccccgcagacatccctga |
| | atgggagcgtgtaatgaattttgaggacggaggggtagtt | ttaccttaaactttcattcccggaagggtttaaatgggagcg |
| | acggtcactcaagatagcagtttacaagatggagagttcat | tgtaatgaattttgaggacggaggggtagttacggtcactc |
| | ttataaggtgaagttacgtggaacaaacttcccgtcagacg | aagatagcagtttacaagatggagagttcatttataaggtg |
| | gtcccgttatgcaaaaaaagacgatgggctgggaggcttc | aagttacgtggaacaaacttcccgtcagacggtcccgttat |
| | ttccgagcgcatgtatccagaagacggtgcactgaaagg | gcaaaaaaagacgatgggctgggaggcttcttccgagcg |
| | cgagatcaagcaacgcctgaaattaaaggacgggggac | catgtatccagaagacggtgcactgaaaggcgagatcaa |
| | attatgacgccgaggtcaaaactacatacaaagctaagaa | gcaacgcctgaaattaaaggacgggggacattatgacgc |
| | acccgttcagttaccaggtgcctacaacgtaaacattaaatt | cgaggtcaaaactacatacaaagctaagaaacccgttcag |
| | ggacattacgtcgcataacgaggattacacgatcgtggaa | ttaccaggtgcctacaacgtaaacattaaattggacattac |
| | caatatgaacgtgctgagggtcgccactccaccgggggt | gtcgcataacgaggattacacgatcgtggaacaatatgaa |
| | atggacgagctttataaataatgagatccggctgctaacaa | cgtgctgagggtcgccactccaccgggggtatggacga |
| | agcccgaaaggaagctgagttggctgctgccaccgctga | gctttataaataatgagatccggctgctaacaaagcccgaa |
| | gcaataactagcataa (SEQ ID NO: 23) | aggaagctgagttggctgctgccaccgctgagcaataact |
| | | agcataacactttagttacaacatacttattgcctcgg |
| | | (SEQ ID NO: 24) |
| 100 | gatgtcggcgatataggcgccagcaaccgcacctgtggc | ggctccgaataagtatgttgtaactaaagtggatgtcggcg |
| | gccggtgatgccggccacgatgcgtccggcgtagagga | atataggcgccagcaaccgcacctgtggcgccggtgatg |
| | tcgagatctcgatcccgcgaaattaatacgactcactatag | ccggccacgatgcgtccggcgtagaggatcgagatctcg |
| | ggagaccacaacggtttccctctagaaataattttgtttaact | atcccgcgaaattaatacgactcactatagggagaccaca |
| | ttaagaaggagatatacatatggtctcaaagggggaagag | acggtttccctctagaaataattttgtttaactttaagaagga |
| | gataatatggcgatcatcaaggagtttatgcgtttcaaagtt | gatatacatatggtctcaaagggggaagaggataatatgg |
| | catatggaaggcagtgttaacggccacgagttcgagattg | cgatcatcaaggagtttatgcgtttcaaagttcatatggaag |
| | agggggagggggaaggacgcccttacgaagggaccca | gcagtgttaacggccacgagttcgagattgagggggagg |
| | aacagctaaattaaaagtgactaaagggggtccgttgcctt | gggaaggacgcccttacgaagggacccaaacagctaaa |
| | tcgcgtgggatatcttatcaccgcagttcatgtatggatcga | ttaaaagtgactaaagggggtccgttgcctttcgcgtggga |
| | aggcttatgtaaaacaccccgcagacatccctgattacctt | tatcttatcaccgcagttcatgtatggatcgaaggcttatgta |
| | aaactttcattcccggaagggtttaaatgggagcgtgtaat | aaacaccccgcagacatccctgattaccttaaactttcattc |
| | gaattttgaggacggaggggtagttacggtcactcaagat | ccggaagggtttaaatgggagcgtgtaatgaattttgagg |
| | agcagtttacaagatggagagttcatttataaggtgaagtta | acggaggggtagttacggtcactcaagatagcagtttaca |
| | cgtggaacaaacttcccgtcagacggtcccgttatgcaaa | agatggagagttcatttataaggtgaagttacgtggaacaa |
| | aaaagacgatgggctgggaggcttcttccgagcgcatgt | acttcccgtcagacggtcccgttatgcaaaaaaagacgat |
| | atccagaagacggtgcactgaaaggcgagatcaagcaa | gggctgggaggcttcttccgagcgcatgtatccagaaga |
| | cgcctgaaattaaaggacgggggacattatgacgccgag | cggtgcactgaaaggcgagatcaagcaacgcctgaaatt |
| | gtcaaaactacatacaaagctaagaaacccgttcagttacc | aaaggacgggggacattatgacgccgaggtcaaaactac |
| | aggtgcctacaacgtaaacattaaattggacattacgtcgc | atacaaagctaagaaacccgttcagttaccaggtgcctac |
| | ataacgaggattacacgatcgtggaacaatatgaacgtgc | aacgtaaacattaaattggacattacgtcgcataacgagga |
| | tgagggtcgccactccaccgggggtatggacgagctttat | ttacacgatcgtggaacaatatgaacgtgctgagggtcgc |
| | aaataatgagatccggctgctaacaaagcccgaaaggaa | cactccaccgggggtatggacgagctttataaataatgag |
| | gctgagttggctgctgccaccgctgagcaataactagcat | atccggctgctaacaaagcccgaaaggaagctgagttgg |
| | aaccccttggggcctctaaacgggtct (SEQ ID NO: | ctgctgccaccgctgagcaataactagcataaccccttgg |
| | | ggcctctaaacgggtctcactttagttacaacatacttattgc |
| | 25) | ctcgg (SEQ ID NO: 26) |
| 200 | tggcgcccaacagtcccccggccacggggcctgccacc | ggctccgaataagtatgttgtaactaaagtgtggcgcccaa |
| | atacccacgccgaaacaagcgctcatgagcccgaagtg | cagtcccccggccacggggcctgccaccatacccacgc |
| | gcgagcccgatcttccccatcggtgatgtcggcgatatag | cgaaacaagcgctcatgagcccgaagtggcgagcccga |
| | gcgccagcaaccgcacctgtggcgccggtgatgccggc | tcttccccatcggtgatgtcggcgatataggcgccagcaa |
| | cacgatgcgtccggcgtagaggatcgagatctcgatccc | ccgcacctgtggcgccggtgatgccggccacgatgcgtc |
| | gcgaaattaatacgactcactatagggagaccacaacggt | cggcgtagaggatcgagatctcgatcccgcgaaattaata |
| | ttccctctagaaataattttgtttaactttaagaaggagatat | cgactcactatagggagaccacaacggtttccctctagaa |
| | acatatggtctcaaagggggaagaggataatatggcgatca | ataattttgtttaactttaagaaggagatatacatatggtctc |
| | tcaaggagtttatgcgtttcaaagttcatatggaaggcagtg | aaagggggaagaggataatatggcgatcatcaaggagttt |
| | ttaacggccacgagttcgagattgagggggagggggaa | atgcgtttcaaagttcatatggaaggcagtgttaacggcca |
| | ggacgcccttacgaagggacccaaacagctaaattaaaa | cgagttcgagattgagggggagggggaaggacgccctt |
| | gtgactaaagggggtccgttgcctttcgcgtgggatatctt | acgaagggacccaaacagctaaattaaaagtgactaaag |
| | atcaccgcagttcatgtatggatcgaaggcttatgtaaaac | ggggtccgttgcctttcgcgtgggatatcttatcaccgcag |
| | accccgcagacatccctgattaccttaaactttcattcccgg | ttcatgtatggatcgaaggcttatgtaaaacaccccgcaga |
| | aagggtttaaatgggagcgtgtaatgaattttgaggacgg | catccctgattaccttaaactttcattcccggaagggtttaaa |
| | aggggtagttacggtcactcaagatagcagtttacaagat | tgggagcgtgtaatgaattttgaggacggaggggtagtta |
| | ggagagttcatttataaggtgaagttacgtggaacaaactt | cggtcactcaagatagcagtttacaagatggagagttcatt |
| | cccgtcagacggtcccgttatgcaaaaaaagacgatggg | tataaggtgaagttacgtggaacaaacttcccgtcagacg |
| | ctgggaggcttcttccgagcgcatgtatccagaagacggt | gtcccgttatgcaaaaaaagacgatgggctgggaggcttc |
| | gcactgaaaggcgagatcaagcaacgcctgaaattaaag | ttccgagcgcatgtatccagaagacggtgcactgaaagg |
| | gacgggggacattatgacgccgaggtcaaaactacatac | cgagatcaagcaacgcctgaaattaaaggacgggggac |
| | aaagctaagaaacccgttcagttaccaggtgcctacaacg | attatgacgccgaggtcaaaactacatacaaagctaagaa |
| | taaacattaaattggacattacgtcgcataacgaggattac | acccgttcagttaccaggtgcctacaacgtaaacattaaatt |
| | acgatcgtggaacaatatgaacgtgctgagggtcgccact | ggacattacgtcgcataacgaggattacacgatcgtggaa |
| | ccaccgggggtatggacgagctttataaataatgagatcc | caatatgaacgtgctgagggtcgccactccaccgggggt |
| | ggctgctaacaaagcccgaaaggaagctgagttggctgc | atggacgagctttataaataatgagatccggctgctaacaa |
| | tgccaccgctgagcaataactagcataaccccttggggcc | agcccgaaaggaagctgagttggctgctgccaccgctga |
| | tctaaacgggtcttgaggggttttttgctgaaaggaggaac | gcaataactagcataaccccttggggcctctaaacgggtc |
| | tatatccggattggcgaatgggacgcgccctgtagcggc | ttgaggggttttttgctgaaaggaggaactatatccggattg |
| | gcattaagcgcggcgggtgtggtggttacgcgc (SEQ | gcgaatgggacgcgccctgtagcggcgcattaagcgcg |
| | ID NO: 27) | gcgggtgtggtggttacgcgccactttagttacaacatactt |
| | | attgcctcgg (SEQ ID NO: 28) |
| 300 | cgacgctctcccttatgcgactcctgcattaggaagcagc | ggctccgaataagtatgttgtaactaaagtgcgacgctctc |
| | ccagtagtaggttgaggccgttgagcaccgccgccgcaa | ccttatgcgactcctgcattaggaagcagcccagtagtag |
| | ggaatggtgcatgcaaggagatggcgcccaacagtccc | gttgaggccgttgagcaccgccgccgcaaggaatggtgc |
| | ccggccacggggcctgccaccatacccacgccgaaaca | atgcaaggagatggcgcccaacagtcccccggccacgg |
| | agcgctcatgagcccgaagtggcgagcccgatcttcccc | ggcctgccaccatacccacgccgaaacaagcgctcatga |
| | atcggtgatgtcggcgatataggcgccagcaaccgcacc | gcccgaagtggcgagcccgatcttccccatcggtgatgtc |
| | tgtggcgccggtgatgccggccacgatgcgtccggcgta | ggcgatataggcgccagcaaccgcacctgtggcgccgg |
| | gaggatcgagatctcgatcccgcgaaattaatacgactca | tgatgccggccacgatgcgtccggcgtagaggatcgaga |
| | ctatagggagaccacaacggtttccctctagaaataattttg | tctcgatcccgcgaaattaatacgactcactatagggagac |
| | tttaactttaagaaggagatatacatatggtctcaaagggg | cacaacggtttccctctagaaataattttgtttaactttaaga |
| | gaagaggataatatggcgatcatcaaggagtttatgcgttt | aggagatatacatatggtctcaaagggggaagaggataata |
| | caaagttcatatggaaggcagtgttaacggccacgagttc | tggcgatcatcaaggagtttatgcgtttcaaagttcatatgg |
| | gagattgagggggagggggaaggacgcccttacgaag | aaggcagtgttaacggccacgagttcgagattgaggggg |
| | ggacccaaacagctaaattaaaagtgactaaagggggtc | agggggaaggacgcccttacgaagggacccaaacagct |
| | cgttgcctttcgcgtgggatatcttatcaccgcagttcatgta | aaattaaaagtgactaaagggggtccgttgcctttcgcgtg |
| | tggatcgaaggcttatgtaaaacaccccgcagacatccct | ggatatcttatcaccgcagttcatgtatggatcgaaggctta |
| | gattaccttaaactttcattcccggaagggtttaaatgggag | tgtaaaacaccccgcagacatccctgattaccttaaactttc |
| | cgtgtaatgaattttgaggacggaggggtagttacggtca | attcccggaagggtttaaatgggagcgtgtaatgaattttg |
| | ctcaagatagcagtttacaagatggagagttcatttataag | aggacggaggggtagttacggtcactcaagatagcagttt |
| | gtgaagttacgtggaacaaacttcccgtcagacggtcccg | acaagatggagagttcatttataaggtgaagttacgtggaa |
| | ttatgcaaaaaaagacgatgggctgggaggcttcttccga | caaacttcccgtcagacggtcccgttatgcaaaaaaagac |
| | gcgcatgtatccagaagacggtgcactgaaaggcgagat | gatgggctgggaggcttcttccgagcgcatgtatccagaa |
| | caagcaacgcctgaaattaaaggacgggggacattatga | gacggtgcactgaaaggcgagatcaagcaacgcctgaa |
| | cgccgaggtcaaaactacatacaaagctaagaaacccgtt | attaaaggacgggggacattatgacgccgaggtcaaaac |
| | cagttaccaggtgcctacaacgtaaacattaaattggacat | tacatacaaagctaagaaacccgttcagttaccaggtgcct |
| | tacgtcgcataacgaggattacacgatcgtggaacaatat | acaacgtaaacattaaattggacattacgtcgcataacgag |
| | gaacgtgctgagggtcgccactccaccgggggtatggac | gattacacgatcgtggaacaatatgaacgtgctgagggtc |
| | gagctttataaataatgagatccggctgctaacaaagccc | gccactccaccgggggtatggacgagctttataaataatg |
| | gaaaggaagctgagttggctgctgccaccgctgagcaat | agatccggctgctaacaaagcccgaaaggaagctgagtt |
| | aactagcataaccccttggggcctctaaacgggtcttgag | ggctgctgccaccgctgagcaataactagcataacccctt |
| | gggttttttgctgaaaggaggaactatatccggattggcga | ggggcctctaaacgggtcttgaggggttttttgctgaaagg |
| | atgggacgcgccctgtagcggcgcattaagcgcggcgg | aggaactatatccggattggcgaatgggacgcgccctgta |
| | gtgtggtggttacgcgcagcgtgaccgctacacttgccag | gcggcgcattaagcgcggcgggtgtggtggttacgcgca |
| | cgccctagcgcccgctcctttcgctttcttcccttcctttctc | gcgtgaccgctacacttgccagcgccctagcgcccgctc |
| | gccacgttcgccggctttccccgtcaagctctaa (SEQ | ctttcgctttcttcccttcctttctcgccacgttcgccggctt |
| | ID NO: 29) | tccccgtcaagctctaacactttagttacaacatacttattg |
| | | cctcgg (SEQ ID NO: 30) |
| 0-125 | taatacgactcactatagggagaccacaacggtttccctct | ggctccgaataagtatgttgtaactaaagtgtaatacgactc |
| | agaaataattttgtttaactttaagaaggagatatacatatgg | actatagggagaccacaacggtttccctctagaaataatttt |
| | tctcaaagggggaagaggataatatggcgatcatcaagg | gtttaactttaagaaggagatatacatatggtctcaaaggg |
| | agtttatgcgtttcaaagttcatatggaaggcagtgttaacg | ggaagaggataatatggcgatcatcaaggagtttatgcgtt |
| | gccacgagttcgagattgagggggagggggaaggacg | tcaaagttcatatggaaggcagtgttaacggccacgagttc |
| | cccttacgaagggacccaaacagctaaattaaaagtgact | gagattgagggggagggggaaggacgcccttacgaag |
| | aaagggggtccgttgcctttcgcgtgggatatcttatcacc | ggacccaaacagctaaattaaaagtgactaaagggggtc |
| | gcagttcatgtatggatcgaaggcttatgtaaaacaccccg | cgttgcctttcgcgtgggatatcttatcaccgcagttcatgta |
| | cagacatccctgattaccttaaactttcattcccggaagggt | tggatcgaaggcttatgtaaaacaccccgcagacatccct |
| | ttaaatgggagcgtgtaatgaattttgaggacggaggggt | gattaccttaaactttcattcccggaagggtttaaatgggag |
| | agttacggtcactcaagatagcagtttacaagatggagagt | cgtgtaatgaattttgaggacggaggggtagttacggtca |
| | tcatttataaggtgaagttacgtggaacaaacttcccgtcag | ctcaagatagcagtttacaagatggagagttcatttataag |
| | acggtcccgttatgcaaaaaaagacgatgggctgggagg | gtgaagttacgtggaacaaacttcccgtcagacggtcccg |
| | cttcttccgagcgcatgtatccagaagacggtgcactgaa | ttatgcaaaaaaagacgatgggctgggaggcttcttccga |
| | aggcgagatcaagcaacgcctgaaattaaaggacgggg | gcgcatgtatccagaagacggtgcactgaaaggcgagat |
| | gacattatgacgccgaggtcaaaactacatacaaagctaa | caagcaacgcctgaaattaaaggacgggggacattatga |
| | gaaacccgttcagttaccaggtgcctacaacgtaaacatta | cgccgaggtcaaaactacatacaaagctaagaaacccgtt |
| | aattggacattacgtcgcataacgaggattacacgatcgtg | cagttaccaggtgcctacaacgtaaacattaaattggacat |
| | gaacaatatgaacgtgctgagggtcgccactccaccggg | tacgtcgcataacgaggattacacgatcgtggaacaatat |
| | ggtatggacgagctttataaataatgagatccggctgctaa | gaacgtgctgagggtcgccactccaccgggggtatggac |
| | caaagcccgaaaggaagctgagttggctgctgccaccgc | gagctttataaataatgagatccggctgctaacaaagccc |
| | tgagcaataactagcataaccccttggggcctctaaacgg | gaaaggaagctgagttggctgctgccaccgctgagcaat |
| | gtcttgaggggttttttgctgaaaggagg (SEQ ID | aactagcataaccccttggggcctctaaacgggtcttgag |
| | NO: 31) | gggttttttgctgaaaggaggcactttagttacaacatactta |
| | | ttgcctcgg (SEQ ID NO: 32) |
| 10-125 | cccgcgaaattaatacgactcactatagggagaccacaac | ggctccgaataagtatgttgtaactaaagtgcccgcgaaat |
| | ggtttccctctagaaataattttgtttaactttaagaaggaga | taatacgactcactatagggagaccacaacggtttccctct |
| | tatacatatggtctcaaagggggaagaggataatatggcga | agaaataattttgtttaactttaagaaggagatatacatatgg |
| | tcatcaaggagtttatgcgtttcaaagttcatatggaaggca | tctcaaagggggaagaggataatatggcgatcatcaagg |
| | gtgttaacggccacgagttcgagattgagggggagggg | agtttatgcgtttcaaagttcatatggaaggcagtgttaacg |
| | gaaggacgcccttacgaagggacccaaacagctaaatta | gccacgagttcgagattgagggggagggggaaggacg |
| | aaagtgactaaagggggtccgttgcctttcgcgtgggatat | cccttacgaagggacccaaacagctaaattaaaagtgact |
| | cttatcaccgcagttcatgtatggatcgaaggcttatgtaaa | aaagggggtccgttgcctttcgcgtgggatatcttatcacc |
| | acaccccgcagacatccctgattaccttaaactttcattccc | gcagttcatgtatggatcgaaggcttatgtaaaacaccccg |
| | ggaagggtttaaatgggagcgtgtaatgaattttgaggac | cagacatccctgattaccttaaactttcattcccggaagggt |
| | ggaggggtagttacggtcactcaagatagcagtttacaag | ttaaatgggagcgtgtaatgaattttgaggacggaggggt |
| | atggagagttcatttataaggtgaagttacgtggaacaaac | agttacggtcactcaagatagcagtttacaagatggagagt |
| | ttcccgtcagacggtcccgttatgcaaaaaaagacgatgg | tcatttataaggtgaagttacgtggaacaaacttcccgtcag |
| | gctgggaggcttcttccgagcgcatgtatccagaagacg | acggtcccgttatgcaaaaaaagacgatgggctgggagg |
| | gtgcactgaaaggcgagatcaagcaacgcctgaaattaa | cttcttccgagcgcatgtatccagaagacggtgcactgaa |
| | aggacgggggacattatgacgccgaggtcaaaactacat | aggcgagatcaagcaacgcctgaaattaaaggacgggg |
| | acaaagctaagaaacccgttcagttaccaggtgcctacaa | gacattatgacgccgaggtcaaaactacatacaaagctaa |
| | cgtaaacattaaattggacattacgtcgcataacgaggatt | gaaacccgttcagttaccaggtgcctacaacgtaaacatta |
| | acacgatcgtggaacaatatgaacgtgctgagggtcgcc | aattggacattacgtcgcataacgaggattacacgatcgtg |
| | actccaccgggggtatggacgagctttataaataatgagat | gaacaatatgaacgtgctgagggtcgccactccaccggg |
| | ccggctgctaacaaagcccgaaaggaagctgagttggct | ggtatggacgagctttataaataatgagatccggctgctaa |
| | gctgccaccgctgagcaataactagcataaccccttgggg | caaagcccgaaaggaagctgagttggctgctgccaccgc |
| | cctctaaacgggtcttgaggggttttttgctgaaaggagg | tgagcaataactagcataaccccttggggcctctaaacgg |
| | (SEQ ID NO: 33) | gtcttgaggggttttttgctgaaaggaggcactttagttaca |
| | | acatacttattgcctcgg (SEQ ID NO: 34) |
| 20-125 | agatctcgatcccgcgaaattaatacgactcactataggga | ggctccgaataagtatgttgtaactaaagtgagatctcgat |
| | gaccacaacggtttccctctagaaataattttgtttaacttta | cccgcgaaattaatacgactcactatagggagaccacaac |
| | agaaggagatatacatatggtctcaaagggggaagaggat | ggtttccctctagaaataattttgtttaactttaagaaggaga |
| | aatatggcgatcatcaaggagtttatgcgtttcaaagttcat | tatacatatggtctcaaagggggaagaggataatatggcga |
| | atggaaggcagtgttaacggccacgagttcgagattgag | tcatcaaggagtttatgcgtttcaaagttcatatggaaggca |
| | ggggagggggaaggacgcccttacgaagggacccaaa | gtgttaacggccacgagttcgagattgagggggagggg |
| | cagctaaattaaaagtgactaaagggggtccgttgcctttc | gaaggacgcccttacgaagggacccaaacagctaaatta |
| | gcgtgggatatcttatcaccgcagttcatgtatggatcgaa | aaagtgactaaagggggtccgttgcctttcgcgtgggatat |
| | ggcttatgtaaaacaccccgcagacatccctgattacctta | cttatcaccgcagttcatgtatggatcgaaggcttatgtaaa |
| | aactttcattcccggaagggtttaaatgggagcgtgtaatg | acaccccgcagacatccctgattaccttaaactttcattccc |
| | aattttgaggacggaggggtagttacggtcactcaagata | ggaagggtttaaatgggagcgtgtaatgaattttgaggac |
| | gcagtttacaagatggagagttcatttataaggtgaagttac | ggaggggtagttacggtcactcaagatagcagtttacaag |
| | gtggaacaaacttcccgtcagacggtcccgttatgcaaaa | atggagagttcatttataaggtgaagttacgtggaacaaac |
| | aaagacgatgggctgggaggcttcttccgagcgcatgtat | ttcccgtcagacggtcccgttatgcaaaaaaagacgatgg |
| | ccagaagacggtgcactgaaaggcgagatcaagcaacg | gctgggaggcttcttccgagcgcatgtatccagaagacg |
| | cctgaaattaaaggacgggggacattatgacgccgaggt | gtgcactgaaaggcgagatcaagcaacgcctgaaattaa |
| | caaaactacatacaaagctaagaaacccgttcagttacca | aggacgggggacattatgacgccgaggtcaaaactacat |
| | ggtgcctacaacgtaaacattaaattggacattacgtcgca | acaaagctaagaaacccgttcagttaccaggtgcctacaa |
| | taacgaggattacacgatcgtggaacaatatgaacgtgct | cgtaaacattaaattggacattacgtcgcataacgaggatt |
| | gagggtcgccactccaccgggggtatggacgagctttat | acacgatcgtggaacaatatgaacgtgctgagggtcgcc |
| | aaataatgagatccggctgctaacaaagcccgaaaggaa | actccaccgggggtatggacgagctttataaataatgagat |
| | gctgagttggctgctgccaccgctgagcaataactagcat | ccggctgctaacaaagcccgaaaggaagctgagttggct |
| | aaccccttggggcctctaaacgggtcttgaggggttttttg | gctgccaccgctgagcaataactagcataaccccttgggg |
| | ctgaaaggagg (SEQ ID NO: 35) | cctctaaacgggtcttgaggggttttttgctgaaaggaggc |
| | | actttagttacaacatacttattgcctcgg (SEQ ID |
| | | NO: 36) |
| 50-125 | cggccacgatgcgtccggcgtagaggatcgagatctcga | ggctccgaataagtatgttgtaactaaagtgcggccacgat |
| | tcccgcgaaattaatacgactcactatagggagaccacaa | gcgtccggcgtagaggatcgagatctcgatcccgcgaaa |
| | cggtttccctctagaaataattttgtttaactttaagaaggag | ttaatacgactcactatagggagaccacaacggtttccctc |
| | atatacatatggtctcaaagggggaagaggataatatggc | tagaaataattttgtttaactttaagaaggagatatacatatg |
| | gatcatcaaggagtttatgcgtttcaaagttcatatggaagg | gtctcaaagggggaagaggataatatggcgatcatcaag |
| | cagtgttaacggccacgagttcgagattgagggggggg | gagtttatgcgtttcaaagttcatatggaaggcagtgttaac |
| | ggaaggacgcccttacgaagggacccaaacagctaaatt | ggccacgagttcgagattgagggggagggggaaggac |
| | aaaagtgactaaagggggtccgttgcctttcgcgtgggat | gcccttacgaagggacccaaacagctaaattaaaagtga |
| | atcttatcaccgcagttcatgtatggatcgaaggcttatgta | ctaaagggggtccgttgcctttcgcgtgggatatcttatcac |
| | aaacaccccgcagacatccctgattaccttaaactttcattc | cgcagttcatgtatggatcgaaggcttatgtaaaacacccc |
| | ccggaagggtttaaatgggagcgtgtaatgaattttgagg | gcagacatccctgattaccttaaactttcattcccggaagg |
| | acggaggggtagttacggtcactcaagatagcagtttaca | gtttaaatgggagcgtgtaatgaattttgaggacggaggg |
| | agatggagagttcatttataaggtgaagttacgtggaacaa | gtagttacggtcactcaagatagcagtttacaagatggaga |
| | acttcccgtcagacggtcccgttatgcaaaaaaagacgat | gttcatttataaggtgaagttacgtggaacaaacttcccgtc |
| | gggctgggaggcttcttccgagcgcatgtatccagaaga | agacggtcccgttatgcaaaaaaagacgatgggctggga |
| | cggtgcactgaaaggcgagatcaagcaacgcctgaaatt | ggcttcttccgagcgcatgtatccagaagacggtgcactg |
| | aaaggacgggggacattatgacgccgaggtcaaaactac | aaaggcgagatcaagcaacgcctgaaattaaaggacgg |
| | atacaaagctaagaaacccgttcagttaccaggtgcctac | gggacattatgacgccgaggtcaaaactacatacaaagct |
| | aacgtaaacattaaattggacattacgtcgcataacgagga | aagaaacccgttcagttaccaggtgcctacaacgtaaaca |
| | ttacacgatcgtggaacaatatgaacgtgctgagggtcgc | ttaaattggacattacgtcgcataacgaggattacacgatc |
| | cactccaccgggggtatggacgagctttataaataatgag | gtggaacaatatgaacgtgctgagggtcgccactccacc |
| | atccggctgctaacaaagcccgaaaggaagctgagttgg | gggggtatggacgagctttataaataatgagatccggctg |
| | ctgctgccaccgctgagcaataactagcataaccccttgg | ctaacaaagcccgaaaggaagctgagttggctgctgcca |
| | ggcctctaaacgggtcttgaggggttttttgctgaaaggag | ccgctgagcaataactagcataaccccttggggcctctaa |
| | g (SEQ ID NO: 37) | acgggtcttgaggggttttttgctgaaaggaggcactttagt |
| | | tacaacatacttattgcctcgg (SEQ ID NO: 38) |
| 0T | taatacgactcactatagggagaccacaacggtttccctct | ggctccgaataagtatgttgtaactaaagtgtaatacgactc |
| | agaaataattttgtttaactttaagaaggagatatacatatgg | actatagggagaccacaacggtttccctctagaaataatttt |
| | tctcaaagggggaagaggataatatggcgatcatcaagg | gtttaactttaagaaggagatatacatatggtctcaaaggg |
| | agtttatgcgtttcaaagttcatatggaaggcagtgttaacg | ggaagaggataatatggcgatcatcaaggagtttatgcgtt |
| | gccacgagttcgagattgagggggagggggaaggacg | tcaaagttcatatggaaggcagtgttaacggccacgagttc |
| | cccttacgaagggacccaaacagctaaattaaaagtgact | gagattgagggggagggggaaggacgcccttacgaag |
| | aaagggggtccgttgcctttcgcgtgggatatcttatcacc | ggacccaaacagctaaattaaaagtgactaaagggggtc |
| | gcagttcatgtatggatcgaaggcttatgtaaaacaccccg | cgttgcctttcgcgtgggatatcttatcaccgcagttcatgta |
| | cagacatccctgattaccttaaactttcattcccggaagggt | tggatcgaaggcttatgtaaaacaccccgcagacatccct |
| | ttaaatgggagcgtgtaatgaattttgaggacggaggggt | gattaccttaaactttcattcccggaagggtttaaatgggag |
| | agttacggtcactcaagatagcagtttacaagatggagagt | cgtgtaatgaattttgaggacggaggggtagttacggtca |
| | tcatttataaggtgaagttacgtggaacaaacttcccgtcag | ctcaagatagcagtttacaagatggagagttcatttataag |
| | acggtcccgttatgcaaaaaaagacgatgggctgggagg | gtgaagttacgtggaacaaacttcccgtcagacggtcccg |
| | cttcttccgagcgcatgtatccagaagacggtgcactgaa | ttatgcaaaaaaagacgatgggctgggaggcttcttccga |
| | aggcgagatcaagcaacgcctgaaattaaaggacgggg | gcgcatgtatccagaagacggtgcactgaaaggcgagat |
| | gacattatgacgccgaggtcaaaactacatacaaagctaa | caagcaacgcctgaaattaaaggacgggggacattatga |
| | gaaacccgttcagttaccaggtgcctacaacgtaaacatta | cgccgaggtcaaaactacatacaaagctaagaaacccgtt |
| | aattggacattacgtcgcataacgaggattacacgatcgtg | cagttaccaggtgcctacaacgtaaacattaaattggacat |
| | gaacaatatgaacgtgctgagggtcgccactccaccggg | tacgtcgcataacgaggattacacgatcgtggaacaatat |
| | ggtatggacgagctttataaataactagcataaccccttgg | gaacgtgctgagggtcgccactccaccgggggtatggac |
| | ggcctctaaacgggtcttgaggggttttttgctgaaaggag | gagctttataaataactagcataaccccttggggcctctaa |
| | g (SEQ ID NO: 39) | acgggtcttgaggggttttttgctgaaaggaggcactttagt |
| | | tacaacatacttattgcctcgg (SEQ ID NO: 40) |
| 10T | cccgcgaaattaatacgactcactatagggagaccacaac | ggctccgaataagtatgttgtaactaaagtgcccgcgaaat |
| | ggtttccctctagaaataattttgtttaactttaagaaggaga | taatacgactcactatagggagaccacaacggtttccctct |
| | tatacatatggtctcaaagggggaagaggataatatggcga | agaaataattttgtttaactttaagaaggagatatacatatgg |
| | tcatcaaggagtttatgcgtttcaaagttcatatggaaggca | tctcaaagggggaagaggataatatggcgatcatcaagg |
| | gtgttaacggccacgagttcgagattgagggggagggg | agtttatgcgtttcaaagttcatatggaaggcagtgttaacg |
| | gaaggacgcccttacgaagggacccaaacagctaaatta | gccacgagttcgagattgagggggagggggaaggacg |
| | aaagtgactaaagggggtccgttgcctttcgcgtgggatat | cccttacgaagggacccaaacagctaaattaaaagtgact |
| | cttatcaccgcagttcatgtatggatcgaaggcttatgtaaa | aaagggggtccgttgcctttcgcgtgggatatcttatcacc |
| | acaccccgcagacatccctgattaccttaaactttcattccc | gcagttcatgtatggatcgaaggcttatgtaaaacaccccg |
| | ggaagggtttaaatgggagcgtgtaatgaattttgaggac | cagacatccctgattaccttaaactttcattcccggaagggt |
| | ggaggggtagttacggtcactcaagatagcagtttacaag | ttaaatgggagcgtgtaatgaattttgaggacggaggggt |
| | atggagagttcatttataaggtgaagttacgtggaacaaac | agttacggtcactcaagatagcagtttacaagatggagagt |
| | ttcccgtcagacggtcccgttatgcaaaaaaagacgatgg | tcatttataaggtgaagttacgtggaacaaacttcccgtcag |
| | gctgggaggcttcttccgagcgcatgtatccagaagacg | acggtcccgttatgcaaaaaaagacgatgggctgggagg |
| | gtgcactgaaaggcgagatcaagcaacgcctgaaattaa | cttcttccgagcgcatgtatccagaagacggtgcactgaa |
| | aggacgggggacattatgacgccgaggtcaaaactacat | aggcgagatcaagcaacgcctgaaattaaaggacgggg |
| | acaaagctaagaaacccgttcagttaccaggtgcctacaa | gacattatgacgccgaggtcaaaactacatacaaagctaa |
| | cgtaaacattaaattggacattacgtcgcataacgaggatt | gaaacccgttcagttaccaggtgcctacaacgtaaacatta |
| | acacgatcgtggaacaatatgaacgtgctgagggtcgcc | aattggacattacgtcgcataacgaggattacacgatcgtg |
| | actccaccgggggtatggacgagctttataaataatgagat | gaacaatatgaacgtgctgagggtcgccactccaccggg |
| | ccggctagcataaccccttggggcctctaaacgggtcttg | ggtatggacgagctttataaataatgagatccggctagcat |
| | aggggttttttgctgaaaggagg (SEQ ID NO: 41) | aaccccttggggcctctaaacgggtcttgaggggttttttg |
| | | ctgaaaggaggcactttagttacaacatacttattgcctcgg |
| | | (SEQ ID NO: 42) |
| 20T | agatctcgatcccgcgaaattaatacgactcactataggga | ggctccgaataagtatgttgtaactaaagtgagatctcgat |
| | gaccacaacggtttccctctagaaataattttgtttaacttta | cccgcgaaattaatacgactcactatagggagaccacaac |
| | agaaggagatatacatatggtctcaaagggggaagaggat | ggtttccctctagaaataattttgtttaactttaagaaggaga |
| | aatatggcgatcatcaaggagtttatgcgtttcaaagttcat | tatacatatggtctcaaagggggaagaggataatatggcga |
| | atggaaggcagtgttaacggccacgagttcgagattgag | tcatcaaggagtttatgcgtttcaaagttcatatggaaggca |
| | ggggagggggaaggacgcccttacgaagggacccaaa | gtgttaacggccacgagttcgagattgagggggagggg |
| | cagctaaattaaaagtgactaaagggggtccgttgcctttc | gaaggacgcccttacgaagggacccaaacagctaaatta |
| | gcgtgggatatcttatcaccgcagttcatgtatggatcgaa | aaagtgactaaagggggtccgttgcctttcgcgtgggatat |
| | ggcttatgtaaaacaccccgcagacatccctgattacctta | cttatcaccgcagttcatgtatggatcgaaggcttatgtaaa |
| | aactttcattcccggaagggtttaaatgggagcgtgtaatg | acaccccgcagacatccctgattaccttaaactttcattccc |
| | aattttgaggacggaggggtagttacggtcactcaagata | ggaagggtttaaatgggagcgtgtaatgaattttgaggac |
| | gcagtttacaagatggagagttcatttataaggtgaagttac | ggaggggtagttacggtcactcaagatagcagtttacaag |
| | gtggaacaaacttcccgtcagacggtcccgttatgcaaaa | atggagagttcatttataaggtgaagttacgtggaacaaac |
| | aaagacgatgggctgggaggcttcttccgagcgcatgtat | ttcccgtcagacggtcccgttatgcaaaaaaagacgatgg |
| | ccagaagacggtgcactgaaaggcgagatcaagcaacg | gctgggaggcttcttccgagcgcatgtatccagaagacg |
| | cctgaaattaaaggacgggggacattatgacgccgaggt | gtgcactgaaaggcgagatcaagcaacgcctgaaattaa |
| | caaaactacatacaaagctaagaaacccgttcagttacca | aggacgggggacattatgacgccgaggtcaaaactacat |
| | ggtgcctacaacgtaaacattaaattggacattacgtcgca | acaaagctaagaaacccgttcagttaccaggtgcctacaa |
| | taacgaggattacacgatcgtggaacaatatgaacgtgct | cgtaaacattaaattggacattacgtcgcataacgaggatt |
| | gagggtcgccactccaccgggggtatggacgagctttat | acacgatcgtggaacaatatgaacgtgctgagggtcgcc |
| | aaataatgagatccggctgctaacaactagcataacccctt | actccaccgggggtatggacgagctttataaataatgagat |
| | ggggcctctaaacgggtcttgaggggttttttgctgaaagg | ccggctgctaacaactagcataaccccttggggcctctaa |
| | agg (SEQ ID NO: 43) | acgggtcttgaggggttttttgctgaaaggaggcactttagt |
| | | tacaacatacttattgcctcgg (SEQ ID NO: 44) |
| 30T | tagaggatcgagatctcgatcccgcgaaattaatacgact | ggctccgaataagtatgttgtaactaaagtgtagaggatcg |
| | cactatagggagaccacaacggtttccctctagaaataatt | agatctcgatcccgcgaaattaatacgactcactataggga |
| | ttgtttaactttaagaaggagatatacatatggtctcaaagg | gaccacaacggtttccctctagaaataattttgtttaacttta |
| | gggaagaggataatatggcgatcatcaaggagtttatgcg | agaaggagatatacatatggtctcaaagggggaagaggat |
| | tttcaaagttcatatggaaggcagtgttaacggccacgagt | aatatggcgatcatcaaggagtttatgcgtttcaaagttcat |
| | tcgagattgagggggagggggaaggacgcccttacgaa | atggaaggcagtgttaacggccacgagttcgagattgag |
| | gggacccaaacagctaaattaaaagtgactaaagggggt | ggggagggggaaggacgcccttacgaagggacccaaa |
| | ccgttgcctttcgcgtgggatatcttatcaccgcagttcatgt | cagctaaattaaaagtgactaaagggggtccgttgcctttc |
| | atggatcgaaggcttatgtaaaacaccccgcagacatccc | gcgtgggatatcttatcaccgcagttcatgtatggatcgaa |
| | tgattaccttaaactttcattcccggaagggtttaaatggga | ggcttatgtaaaacaccccgcagacatccctgattacctta |
| | gcgtgtaatgaattttgaggacggaggggtagttacggtc | aactttcattcccggaagggtttaaatgggagcgtgtaatg |
| | actcaagatagcagtttacaagatggagagttcatttataag | aattttgaggacggaggggtagttacggtcactcaagata |
| | gtgaagttacgtggaacaaacttcccgtcagacggtcccg | gcagtttacaagatggagagttcatttataaggtgaagttac |
| | ttatgcaaaaaaagacgatgggctgggaggcttcttccga | gtggaacaaacttcccgtcagacggtcccgttatgcaaaa |
| | gcgcatgtatccagaagacggtgcactgaaaggcgagat | aaagacgatgggctgggaggcttcttccgagcgcatgtat |
| | caagcaacgcctgaaattaaaggacgggggacattatga | ccagaagacggtgcactgaaaggcgagatcaagcaacg |
| | cgccgaggtcaaaactacatacaaagctaagaaacccgtt | cctgaaattaaaggacgggggacattatgacgccgaggt |
| | cagttaccaggtgcctacaacgtaaacattaaattggacat | caaaactacatacaaagctaagaaacccgttcagttacca |
| | tacgtcgcataacgaggattacacgatcgtggaacaatat | ggtgcctacaacgtaaacattaaattggacattacgtcgca |
| | gaacgtgctgagggtcgccactccaccgggggtatggac | taacgaggattacacgatcgtggaacaatatgaacgtgct |
| | gagctttataaataatgagatccggctgctaacaaagccc | gagggtcgccactccaccgggggtatggacgagctttat |
| | gaaagctagcataaccccttggggcctctaaacgggtctt | aaataatgagatccggctgctaacaaagcccgaaagcta |
| | gaggggttttttgctgaaaggagg (SEQ ID NO: | gcataaccccttggggcctctaaacgggtcttgaggggttt |
| | 45) | tttgctgaaaggaggcactttagttacaacatacttattgcct |
| | | cgg (SEQ ID NO: 46) |
| 50T | cggccacgatgcgtccggcgtagaggatcgagatctcga | ggctccgaataagtatgttgtaactaaagtgcggccacgat |
| | tcccgcgaaattaatacgactcactatagggagaccacaa | gcgtccggcgtagaggatcgagatctcgatcccgcgaaa |
| | cggtttccctctagaaataattttgtttaactttaagaaggag | ttaatacgactcactatagggagaccacaacggtttccctc |
| | atatacatatggtctcaaagggggaagaggataatatggc | tagaaataattttgtttaactttaagaaggagatatacatatg |
| | gatcatcaaggagtttatgcgtttcaaagttcatatggaagg | gtctcaaagggggaagaggataatatggcgatcatcaag |
| | cagtgttaacggccacgagttcgagattgagggggaggg | gagtttatgcgtttcaaagttcatatggaaggcagtgttaac |
| | ggaaggacgcccttacgaagggacccaaacagctaaatt | ggccacgagttcgagattgagggggagggggaaggac |
| | aaaagtgactaaagggggtccgttgcctttcgcgtgggat | gcccttacgaagggacccaaacagctaaattaaaagtga |
| | atcttatcaccgcagttcatgtatggatcgaaggcttatgta | ctaaagggggtccgttgcctttcgcgtgggatatcttatcac |
| | aaacaccccgcagacatccctgattaccttaaactttcattc | cgcagttcatgtatggatcgaaggcttatgtaaaacacccc |
| | ccggaagggtttaaatgggagcgtgtaatgaattttgagg | gcagacatccctgattaccttaaactttcattcccggaagg |
| | acggaggggtagttacggtcactcaagatagcagtttaca | gtttaaatgggagcgtgtaatgaattttgaggacggaggg |
| | agatggagagttcatttataaggtgaagttacgtggaacaa | gtagttacggtcactcaagatagcagtttacaagatggaga |
| | acttcccgtcagacggtcccgttatgcaaaaaaagacgat | gttcatttataaggtgaagttacgtggaacaaacttcccgtc |
| | gggctgggaggcttcttccgagcgcatgtatccagaaga | agacggtcccgttatgcaaaaaaagacgatgggctggga |
| | cggtgcactgaaaggcgagatcaagcaacgcctgaaatt | ggcttcttccgagcgcatgtatccagaagacggtgcactg |
| | aaaggacgggggacattatgacgccgaggtcaaaactac | aaaggcgagatcaagcaacgcctgaaattaaaggacgg |
| | atacaaagctaagaaacccgttcagttaccaggtgcctac | gggacattatgacgccgaggtcaaaactacatacaaagct |
| | aacgtaaacattaaattggacattacgtcgcataacgagga | aagaaacccgttcagttaccaggtgcctacaacgtaaaca |
| | ttacacgatcgtggaacaatatgaacgtgctgagggtcgc | ttaaattggacattacgtcgcataacgaggattacacgatc |
| | cactccaccgggggtatggacgagctttataaataatgag | gtggaacaatatgaacgtgctgagggtcgccactccacc |
| | atccggctgctaacaaagcccgaaaggaagctgagttgg | gggggtatggacgagctttataaataatgagatccggctg |
| | ctgctgcctagcataaccccttggggcctctaaacgggtct | ctaacaaagcccgaaaggaagctgagttggctgctgcct |
| | tgaggggttttttgctgaaaggagg (SEQ ID NO: | agcataaccccttggggcctctaaacgggtcttgaggggt |
| | 47) | tttttgctgaaaggaggcactttagttacaacatacttattgc |
| | | ctcgg (SEQ ID NO: 48) |

Primer Sequences

| | | SEQ | |
|---|---|---|---|
| | primer | ID | |
| | name | NO | |
| minus | 300 fw | 49 | cgacgctctcccttatgcgactcctg |
| ter | 200 fw | 50 | tggcgcccaacagtcccccggccac |
| | 175 fw | 51 | ggggcctgccaccatacccacgc |
| | 150 fw | 52 | aaacaagcgctcatgagcccgaagtg |
| | 125 fw | 53 | ggcgagcccgatcttccccatcgg |
| | 100 fw | 54 | gatgtcggcgatataggcgccagcaac |
| | 75 fw | 55 | accgcacctgtggcgccggtgatgc |
| | 50 fw | 56 | cggccacgatgcgtccggcgtag |
| | 40 fw | 57 | gcgtccggcgtagaggatcgagatc |
| | 30 fw | 58 | tagaggatcgagatctcgatcccg |
| | 20 fw | 59 | agatctcgatcccgcgaaattaatacg |
| | 10 fw | 60 | cccgcgaaattaatacgactcactataggg |
| | 0 fw | 61 | taatacgactcactatagggagacc |
| | 300 rv | 62 | ttagagcttgacggggaaagccggc |
| | 200 rv | 63 | gcgcgtaaccaccacacccgccgc |
| | 175 rv | 64 | cttaatgcgccgctacagggcgcg |
| | 150 rv | 65 | cccattcgccaatccggatatagttcc |
| | 125 rv | 66 | cctcctttcagcaaaaaacccctcaagac |
| | 100 rv | 67 | agacccgtttagaggccccaaggggt |
| | 75 rv | 68 | ttatgctagttattgctcagcggtggc |
| | 50 rv | 69 | gcagcagccaactcagcttcctttcgg |
| | 40 rv | 70 | actcagcttcctttcgggctttgttag |
| | 30 rv | 71 | ctttcgggctttgttagcagccggatc |
| | 20 rv for | 72 | ttgttagcagccggatctcattatttataaagc |
| | mcherry | | |
| | 20 rv for | 73 | ttgttagcagccggatctcattagatccc |
| | degfp | | |
| | 10 rv for | 74 | ccggatctcattatttataaagctcgtcc |
| | mcherry | | |
| | 10 rv for | 75 | ccggatctcattagatcccggcggcggtc |
| | degfp | | |
| | 0 rv for | 76 | ttatttataaagctcgtccatacccccggtg |
| | mcherry | | |
| | 0 rv for | 77 | ttagatcccggcggcggtcacgaactcc |
| | degfp | | |
| | t0 rv for | 78 | cctcctttcagcaaaaaacccctcaagacccgtttagaggccccaaggggttatgctagttatt |
| | mcherry | | tataaagctcgtccatacccccggtg |
| | t0 rv for | 79 | cctcctttcagcaaaaaacccctcaagacccgtttagaggccccaaggggttatgctagttag |
| | degfp | | atcccggcggcggtcacgaactcc |
| | t10 rv | 80 | cctcctttcagcaaaaaacccctcaagacccgtttagaggccccaaggggttatgctagccg |
| | for | | gatctcattatttataaagctcgtcc |
| | mcherry | | |
| | t10 rv | 81 | cctcctttcagcaaaaaacccctcaagacccgtttagaggccccaaggggttatgctagccg |
| | for | | gatctcattagatcccggcggcggtc |
| | degfp | | |
| | t20 rv | 82 | cctcctttcagcaaaaaacccctcaagacccgtttagaggccccaaggggttatgctagttgt |
| | for | | tagcagccggatctcattatttataaagc |
| | mcherry | | |
| | t20 rv | 83 | cctcctttcagcaaaaaacccctcaagacccgtttagaggccccaaggggttatgctagttgt |
| | for | | tagcagccggatctcattagatccc |
| | degfp | | |
| | t30 rv | 84 | cctcctttcagcaaaaaacccctcaagacccgtttagaggccccaaggggttatgctagcttt |
| | | | cgggctttgttagcagccggatc |
| | t50 rv | 85 | cctcctttcagcaaaaaacccctcaagacccgtttagaggccccaaggggttatgctaggca |
| | | | gcagccaactcagcttcctttcgg |
| plus | 300 fw | 86 | ggctccgaataagtatgttgtaactaaagtgcgacgctctcccttatgcgactcctg |
| ter | 200 fw | 87 | ggctccgaataagtatgttgtaactaaagtgtggcgcccaacagtcccccggccac |
| | 175 fw | 88 | ggctccgaataagtatgttgtaactaaagtgggggcctgccaccatacccacgc |
| | 150 fw | 89 | ggctccgaataagtatgttgtaactaaagtgaaacaagcgctcatgagcccgaagtg |
| | 125 fw | 90 | ggctccgaataagtatgttgtaactaaagtgggcgagcccgatcttccccatcgg |
| | 100 fw | 91 | ggctccgaataagtatgttgtaactaaagtggatgtcggcgatataggcgccagcaac |
| | 75 fw | 92 | ggctccgaataagtatgttgtaactaaagtgaccgcacctgtggcgccggtgatgc |
| | 50 fw | 93 | ggctccgaataagtatgttgtaactaaagtgcggccacgatgcgtccggcgtag |
| | 40 fw | 94 | ggctccgaataagtatgttgtaactaaagtggcgtccggcgtagaggatcgagatc |
| | 30 fw | 95 | ggctccgaataagtatgttgtaactaaagtgtagaggatcgagatctcgatcccg |
| | 20 fw | 96 | ggctccgaataagtatgttgtaactaaagtgagatctcgatcccgcgaaattaatacg |
| | 10 fw | 97 | ggctccgaataagtatgttgtaactaaagtgcccgcgaaattaatacgactcactataggg |
| | 0 fw | 98 | ggctccgaataagtatgttgtaactaaagtgtaatacgactcactatagggagacc |
| | 300 rv | 99 | ccgaggcaataagtatgttgtaactaaagtgttagagcttgacggggaaagccggc |
| | 200 rv | 100 | ccgaggcaataagtatgttgtaactaaagtggcgcgtaaccaccacacccgccgc |
| | 175 rv | 101 | ccgaggcaataagtatgttgtaactaaagtgcttaatgcgccgctacagggcgcg |
| | 150 rv | 102 | ccgaggcaataagtatgttgtaactaaagtgcccattcgccaatccggatatagttcc |
| | 125 rv | 103 | ccgaggcaataagtatgttgtaactaaagtgcctcctttcagcaaaaaacccctcaagac |
| | 100 rv | 104 | ccgaggcaataagtatgttgtaactaaagtgagacccgtttagaggccccaaggggt |
| | 75 rv | 105 | ccgaggcaataagtatgttgtaactaaagtgttatgctagttattgctcagcggtggc |
| | 50 rv | 106 | ccgaggcaataagtatgttgtaactaaagtggcagcagccaactcagcttcctttcgg |
| | 40 rv | 107 | ccgaggcaataagtatgttgtaactaaagtgactcagcttcctttcgggctttgttag |
| | 30 rv | 108 | ccgaggcaataagtatgttgtaactaaagtgctttcgggctttgttagcagccggatc |
| | 20 rv for | 109 | ccgaggcaataagtatgttgtaactaaagtgttgttagcagccggatctcattatttataaagc |
| | mcherry | | |
| | 20 rv for | 110 | ccgaggcaataagtatgttgtaactaaagtgttgttagcagccggatctcattagatccc |
| | degfp | | |
| | 10 rv for | 111 | ccgaggcaataagtatgttgtaactaaagtgccggatctcattatttataaagctcgtcc |
| | mcherry | | |
| | 10 rv for | 112 | ccgaggcaataagtatgttgtaactaaagtgccggatctcattagatcccggcggcggtc |
| | degfp | | |
| | 0 rv for | 113 | ccgaggcaataagtatgttgtaactaaagtgttatttataaagctcgtccatacccccggtg |
| | mcherry | | |
| | 0 rv for | 114 | ccgaggcaataagtatgttgtaactaaagtgttagatcccggcggcggtcacgaactcc |
| | degfp | | |
| | t0 rv for | 115 | ccgaggcaataagtatgttgtaactaaagtgcctcctttcagcaaaaaacccctcaagacccg |
| | mcherry | | tttagaggccccaaggggttatgctagttatttataaagctcgtccatacccccggtg |
| | t0 rv for | 116 | ccgaggcaataagtatgttgtaactaaagtgcctcctttcagcaaaaaacccctcaagacccg |
| | degfp | | tttagaggccccaaggggttatgctagttagatcccggcggcggtcacgaactcc |
| | t10 rv | 117 | ccgaggcaataagtatgttgtaactaaagtgcctcctttcagcaaaaaacccctcaagacccg |
| | for | | tttagaggccccaaggggttatgctagccggatctcattatttataaagctcgtcc |
| | mcherry | | |
| | t10 rv | 118 | ccgaggcaataagtatgttgtaactaaagtgcctcctttcagcaaaaaacccctcaagacccg |
| | for | | tttagaggccccaaggggttatgctagccggatctcattagatcccggcggcggtc |
| | degfp | | |
| | t20 rv | 119 | |
| | for | | tttagaggccccaaggggttatgctagttgttagcagccggatctcattatttataaagc |
| | mcherry | | |
| | t20 rv | 120 | ccgaggcaataagtatgttgtaactaaagtgcctcctttcagcaaaaaacccctcaagacccg |
| | for | | tttagaggccccaaggggttatgctagttgttagcagccggatctcattagatccc |
| | degfp | | |
| | t30 rv | 121 | ccgaggcaataagtatgttgtaactaaagtgcctcctttcagcaaaaaacccctcaagacccg |
| | | | tttagaggccccaaggggttatgctagctttcgggctttgttagcagccggatc |
| | t50 rv | 122 | ccgaggcaataagtatgttgtaactaaagtgcctcctttcagcaaaaaacccctcaagacccg |
| | | | tttagaggccccaaggggttatgctaggcagcagccaactcagcttcctttcgg |

Ter DNA sequence
SEQ ID NO: 1: 5′-aataagtatgttgtaactaaagtg-3′
SEQ ID NO: 2: TER 5′ BLOCK (to be placed upstream of the DNA sequence encoding the
protein of interest): aataagtatgttgtaactaaagtg
SEQ ID NO: 3: TER 3′ BLOCK (to be placed downstream of protein of interest):
cactttagttacaacatacttatt
SEQ ID NO: 4: 5′ aataagtatgttgtaactaaagtg----DNA sequence encoding the poi----
cactttagttacaacatacttatt 3′
Tus DNA sequence (SEQ ID NO: 5)
atggcgcgttacgatctcgtagaccgactcaacactacctttcgccagatggaacaagagctggctatatttgccgctcatcttgagcaac
acaagctattggttgcccgcgtgttctctttgccggaggtaaaaaaagaggatgagcataatccgcttaatcgtattgaggtaaaacaaca
tctcggcaacgacgcgcagtcgctggcgttgcgtcatttccgccatttatttattcaacaacagtccgaaaatcgcagcagcaaggccgc
tgtccgtctgcctggcgtgttgtgttaccaggtcgataacctttcgcaagcagcgttggtcagtcatattcagcacatcaataaactcaaga
ccacgttcgagcatatcgtcacggttgaatcagaactccccaccgcggcacgttttgaatgggtgcatcgtcatttgccggggctgatca
cccttaatgcttaccgcacgctcaccgttctgcacgaccccgccactttacgctttggttgggctaataaacatatcattaagaatttacatc
gtgatgaagtcctggcacagctggaaaaaagcctgaaatcaccacgcagtgtcgcaccgtggacgcgcgaggagtggcaaagaaaa
ctggagcgagagtatcaggatatcgctgccctgccacagaacgcgaagttaaaaatcaaacgtccggtgaaggtgcagccgattgccc
gcgtctggtacaaaggagatcaaaaacaagtccaacacgcctgccctacaccactgattgcactgattaatcgggataatggcgcggg
cgtgccggacgttggtgagttgttaaattacgatgccgacaatgtgcagcaccgttataaacctcaggcgcagccgcttcgtttgatcattc
cacggctgcacctgtatgttgcagattaa
Tus Protein sequence (SEQ ID NO: 6)
MARYDLVDRLNTTFRQMEQELAIFAAHLEQHKLLVARVFSLPEVKKEDEHNPLNRIE
VKQHLGNDAQSLALRHFRHLFIQQQSENRSSKAAVRLPGVLCYQVDNLSQAALVSHI
QHINKLKTTFEHIVTVESELPTAARFEWVHRHLPGLITLNAYRTLTVLHDPATLRFGW
ANKHIIKNLHRDEVLAQLEKSLKSPRSVAPWTREEWQRKLEREYQDIAALPQNAKLKI
KRPVKVQPIARVWYKGDQKQVQHACPTPLIALINRDNGAGVPDVGELLNYDADNVQ
HRYKPQAQPLRLIIPRLHLYVAD

1. A linear double stranded deoxyribonucleic acid (dsDNA) molecule comprising operatively linked in the 5′ to 3′ direction:
a) one or more Ter sites at the 5′ terminus (“5′ Ter”);
b) a segment comprising a DNA sequence of interest; and
c) one or more Ter sites at the 3′ terminus (“3′ Ter).

2. The linear dsDNA molecule of claim 1, wherein the DNA sequence of interest is a functional DNA sequence.

3. (canceled)

4. The linear dsDNA molecule of claim 1, wherein the 3′ Ter is downstream a terminator sequence, the DNA sequence of interest is a coding sequence for encoding an expression product and the terminator sequence is located after a STOP codon of the DNA coding sequence and before the 3′ Ter.

5. (canceled)

6. The linear dsDNA molecule of claim 1, wherein the linear dsDNA molecule further comprises a 5′ DNA buffer region upstream the 5′ end of the DNA sequence of interest and a 3′ DNA buffer region 3′ end downstream the DNA sequence of interest, and wherein the 5′ DNA buffer region includes between 0 to 300 base pairs and the 3′ DNA buffer region includes between 0 to 125 base pairs.

7. (canceled)

8. The linear dsDNA molecule of claim 1, wherein the linear dsDNA further comprises a Tus protein bound to the 5′ Ter site and another Tus protein bound to the 3′ Ter.

9. The linear dsDNA molecule of claim 1, wherein at least one of the one or more Ter sites comprises SEQ ID NO:1.

10. The linear dsDNA molecule of claim 1, wherein the one or more Ter sites at the 5′ terminus comprises SEQ ID NO: 2 and the one or more Ter sites at the 3′ terminus comprises SEQ ID NO: 3.

11. A method of protecting a linear deoxyribonucleic acid (DNA) molecule having a free 5′ terminus and a free 3′ terminus from exonuclease degradation comprising:
a) adding one or more Ter sites at the free 5′ terminus (“5′ Ter) of the DNA molecule and adding one or more Ter sites at the 3′ terminus (“3′ Ter”) of the DNA molecule, and
b) binding a Tus protein to each of the 5′ Ter and the 3′ Ter.

12. The method of claim 11, wherein the DNA molecule is a double stranded deoxyribonucleic acid (DNA) molecule.

13. The method of claim 11, wherein the exonuclease is a bacterial exonuclease.

14. The method of claim 11, wherein the DNA molecule includes a functional DNA molecule.

15. (canceled)

16. The method of claim 11, wherein the DNA molecule includes a terminator sequence and the 3′ Ter site is downstream the terminator sequence, the DNA molecule includes a coding sequence for encoding an expression product and the terminator sequence is located after a STOP codon of the DNA molecule coding sequence and before the 3′ Ter.

17. (canceled)

18. The method of claim 11, wherein the method further comprises adding a 5′ DNA buffer region upstream the 5′ end of the DNA molecule and a 3′ DNA buffer region 3′ end downstream of the DNA molecule, and wherein the 5′ DNA buffer region includes between 0 to 300 base pairs and the 3′ DNA buffer region includes between 0 to 125 base pairs.

19. (canceled)

20. The method of claim 11, wherein the Tus is provided as purified Tus or as a Tus-expressing bacterial strain.

21. The method of claim 11, wherein the Tus is provided as a Tus-expressing bacterial strain under control of an endogenous bacterial RNA polymerase.

22. The method of claim 11, wherein at least one of the one or more Ter sites comprises SEQ ID NO:1.

23. The method of claim 11, wherein at least one of the one or more Ter sites at the 5′ terminus comprises SEQ ID NO: 2 and the one or more Ter sites at the 3′ terminus comprises SEQ ID NO: 3.

24. A method of synthesizing a polypeptide of interest in a cell-free protein synthesis (CFPS) reaction mixture comprising:
a) providing the linear dsDNA molecule of claim 1, wherein the DNA sequence of interest is a coding sequence for encoding the polypeptide of interest,
b) providing a Tus protein, and
c) adding the linear dsDNA and the Tus protein to the CFPS, thereby synthesizing the polypeptide of interest.

25. The method of claim 24, wherein the 3′ Ter is downstream a terminator sequence located after a STOP codon of the DNA coding sequence and before the 3′ Ter.

26. (canceled)

27. The method of claim 24, wherein the linear dsDNA molecule further comprises a 5′ DNA buffer region upstream the 5′ end of the DNA sequence of interest and a 3′ DNA buffer region 3′ end downstream the DNA sequence of interest, and wherein the 5′ DNA buffer region includes between 0 to 300 base pairs and the 3′ DNA buffer region includes between about 45 to about 125 base pairs.

28. The method of claim 24, wherein the Tus protein is provided as a purified Tus protein or as a Tus-expressing bacterial strain.

29. The method of claim 24, wherein the CFPS includes a bacteriophage RNA polymerase.

30. (canceled)

31. The method of claim 24, wherein the CFPS is an E. coli lysate-based protein expression having endogenous E. coli RNA polymerase.

32. (canceled)

33. The method of claim 24, wherein at least one of the one or more Ter sites comprises SEQ ID NO: 1.

34. The method of claim 24, wherein at least one of the one or more Ter sites at the 5′ terminus comprises SEQ ID NO: 2 and the one or more Ter sites at the 3′ terminus comprises SEQ ID NO: 3.

35. A cell transformed with the linear dsDNA of claim 1.

36. (canceled)

37. A cell-free synthetic biology system comprising the linear dsDNA molecule of claim 1.

38. The cell-free synthetic biology system of claim 37, wherein the cell-free synthetic biology system comprises an E. coli lysate, a V. natriegens lysate or a B. subtilis lysate.

39. (canceled)


SEQUENCE LISTING (124 sequences)

SEQ ID NO: 1 - DNA, 24 bp, Artificial Sequence
  Synthetic
aataagtatgttgtaactaaagtg

SEQ ID NO: 2 - DNA, 24 bp, Artificial Sequence
  Synthetic
aataagtatgttgtaactaaagtg

SEQ ID NO: 3 - DNA, 24 bp, Artificial Sequence
  Synthetic
cactttagttacaacatacttatt

SEQ ID NO: 4 - DNA, 48 bp, Artificial Sequence
  Synthetic
aataagtatgttgtaactaaagtgcactttagttacaacatacttatt

SEQ ID NO: 5 - DNA, 930 bp, Artificial Sequence
  Synthetic
atggcgcgttacgatctcgtagaccgactcaacactacctttcgccagatggaacaagagctggctatatttgccgctcatcttgagcaacacaagctattggttgcccgcgtgttctctttgccggaggtaaaaaaagaggatgagcataatccgcttaatcgtattgaggtaaaacaacatctcggcaacgacgcgcagtcgctggcgttgcgtcatttccgccatttatttattcaacaacagtccgaaaatcgcagcagcaaggccgctgtccgtctgcctggcgtgttgtgttaccaggtcgataacctttcgcaagcagcgttggtcagtcatattcagcacatcaataaactcaagaccacgttcgagcatatcgtcacggttgaatcagaactccccaccgcggcacgttttgaatgggtgcatcgtcatttgccggggctgatcacccttaatgcttaccgcacgctcaccgttctgcacgaccccgccactttacgctttggttgggctaataaacatatcattaagaatttacatcgtgatgaagtcctggcacagctggaaaaaagcctgaaatcaccacgcagtgtcgcaccgtggacgcgcgaggagtggcaaagaaaactggagcgagagtatcaggatatcgctgccctgccacagaacgcgaagttaaaaatcaaacgtccggtgaaggtgcagccgattgcccgcgtctggtacaaaggagatcaaaaacaagtccaacacgcctgccctacaccactgattgcactgattaatcgggataatggcgcgggcgtgccggacgttggtgagttgttaaattacgatgccgacaatgtgcagcaccgttataaacctcaggcgcagccgcttcgtttgatcattccacggctgcacctgtatgttgcagattaa

SEQ ID NO: 6 - protein, 309 aa, Artificial Sequence [sequence omitted]

SEQ ID NO: 7 - DNA, 2475 bp, Artificial Sequence
  Synthetic
atgcatcaccatcaccatcacaacaaagaaattttggctgtagttgaagccgtatccaatgaaaaggcgctacctcgcgagaagattttcgaagcattggaaagcgcgctggcgacagcaacaaagaaaaaatatgaacaagagatcgacgtccgcgtacagatcgatcgcaaaagcggtgattttgacactttccgtcgctggttagttgttgatgaagtcacccagccgaccaaggaaatcacccttgaagccgcacgttatgaagatgaaagcctgaacctgggcgattacgttgaagatcagattgagtctgttacctttgaccgtatcactacccagacggcaaaacaggttatcgtgcagaaagtgcgtgaagccgaacgtgcgatggtggttgatcagttccgtgaacacgaaggtgaaatcatcaccggcgtggtgaaaaaagtaaaccgcgacaacatctctctggatctgggcaacaacgctgaagccgtgatcctgcgcgaagatatgctgccgcgtgaaaacttccgccctggcgaccgcgttcgtggcgtgctctattccgttcgcccggaagcgcgtggcgcgcaactgttcgtcactcgttccaagccggaaatgctgatcgaactgttccgtattgaagtgccagaaatcggcgaagaagtgattgaaattaaagcagcggctcgcgatccgggttctcgtgcgaaaatcgcggtgaaaaccaacgataaacgtatcgatccggtaggtgcttgcgtaggtatgcgtggcgcgcgtgttcaggcggtgtctactgaactgggtggcgagcgtatcgatatcgtcctgtgggatgataacccggcgcagttcgtgattaacgcaatggcaccggcagacgttgcttctatcgtggtggatgaagataaacacaccatggatatcgccgttgaagccggtaacctggcgcaggcgattggccgtaacggtcagaacgtgcgtctggcttcgcagctgagcggttgggaactcaacgtgatgaccgttgacgacctgcaggctaagcatcaggcggaagcgcacgcagcgatcgacaccttcaccaaatatctcgacatcgacgaagacttcgcgactgttctggtagaagaaggcttctcgacgctggaagaattggcctatgtgccgatgaaagagctgttggaaatcgaaggccttgatgagccgaccgttgaagcactgcgcgagcgtgctaaaaatgcactggccaccattgcacaggcccaggaagaaagcctcggtgataacaaaccggctgacgatctgctgaaccttgaaggggtagatcgtgatttggcattcaaactggccgcccgtggcgtttgtacgctggaagatctcgccgaacagggcattgatgatctggctgatatcgaagggttgaccgacgaaaaagccggagcactgattatggctgcccgtaacatttgctggttcggtgacgaagcggattacgacatcccaacgaccgaaaacctgtattttcagggcatggcgcgttacgatctcgtagaccgactcaacactacctttcgccagatggaacaagagctggctatatttgccgctcatcttgagcaacacaagctattggttgcccgcgtgttctctttgccggaggtaaaaaaagaggatgagcataatccgcttaatcgtattgaggtaaaacaacatctcggcaacgacgcgcagtcgctggcgttgcgtcatttccgccatttatttattcaacaacagtccgaaaatcgcagcagcaaggccgctgtccgtctgcctggcgtgttgtgttaccaggtcgataacctttcgcaagcagcgttggtcagtcatattcagcacatcaataaactcaagaccacgttcgagcatatcgtcacggttgaatcagaactccccaccgcggcacgttttgaatgggtgcatcgtcatttgccggggctgatcacccttaatgcttaccgcacgctcaccgttctgcacgaccccgccactttacgctttggttgggctaataaacatatcattaagaatttacatcgtgatgaagtcctggcacagctggaaaaaagcctgaaatcaccacgcagtgtcgcaccgtggacgcgcgaggagtggcaaagaaaactggagcgagagtatcaggatatcgctgccctgccacagaacgcgaagttaaaaatcaaacgtccggtgaaggtgcagccgattgcccgcgtctggtacaaaggagatcaaaaacaagtccaacacgcctgccctacaccactgattgcactgattaatcgggataatggcgcgggcgtgccggacgttggtgagttgttaaattacgatgccgacaatgtgcagcaccgttataaacctcaggcgcagccgcttcgtttgatcattccacggctgcacctgtatgttgcagattaa

SEQ ID NO: 8 - DNA, 711 bp, Artificial Sequence
  Synthetic
atggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataa

SEQ ID NO: 9 - DNA, 678 bp, Artificial Sequence
  Synthetic
atggagcttttcactggcgttgttcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatctaa

SEQ ID NO: 10 - DNA, 2715 bp, Artificial Sequence
  Synthetic
atgcatcaccatcaccatcacgattacgacatcccaacgaccgaaaacctgtattttcagggcatgaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacaaggctcgcaagtctcgccgtatcagccttgagttcatgcttgagcaagccaataagtttgctaaccataaggccatctggttcccttacaacatggactggcgcggtcgtgtttacgctgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgaaaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggcttacggtgttactcgcagtgtgactaagcgttcagtcatgacgctggcttacgggtccaaagagttcggcttccgtcaacaagtgctggaagataccattcagccagctattgattccggcaagggtctgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagacgcgcttgaacctgatgttcctcggtcagttccgcttacagcctaccattaacaccaacaaagatagcgagattgatgcacacaaacaggagtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 11 - DNA, 791 bp, Artificial Sequence
  Synthetic
taatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataa

SEQ ID NO: 12 - DNA, 853 bp, Artificial Sequence
  Synthetic
ggctccgaataagtatgttgtaactaaagtgtaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataacactttagttacaacatacttattgcctcgg

SEQ ID NO: 13 - DNA, 811 bp, Artificial Sequence
  Synthetic
cccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccgg

SEQ ID NO: 14 - DNA, 873 bp, Artificial Sequence
  Synthetic
ggctccgaataagtatgttgtaactaaagtgcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggcactttagttacaacatacttattgcctcgg

SEQ ID NO: 15 - DNA, 831 bp, Artificial Sequence
  Synthetic
agatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaa

SEQ ID NO: 16 - DNA, 893 bp, Artificial Sequence
  Synthetic
ggctccgaataagtatgttgtaactaaagtgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaacactttagttacaacatacttattgcctcgg

SEQ ID NO: 17 - DNA, 851 bp, Artificial Sequence
  Synthetic
tagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaag

SEQ ID NO: 18 - DNA, 913 bp, Artificial Sequence
  Synthetic
ggctccgaataagtatgttgtaactaaagtgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaagcactttagttacaacatacttattgcctcgg

SEQ ID NO: 19 - DNA, 871 bp, Artificial Sequence
  Synthetic
gcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagt

SEQ ID NO: 20 - DNA, 933 bp, Artificial Sequence
  Synthetic
ggctccgaataagtatgttgtaactaaagtggcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagtcactttagttacaacatacttattgcctcgg

SEQ ID NO: 21 - DNA, 891 bp, Artificial Sequence
  Synthetic
cggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgc

SEQ ID NO: 22 - DNA, 953 bp, Artificial Sequence
  Synthetic
ggctccgaataagtatgttgtaactaaagtgcggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccactttagttacaacatacttattgcctcgg

SEQ ID NO: 23 - DNA, 941 bp, Artificial Sequence
  Synthetic
accgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataa

SEQ ID NO: 24 - DNA, 1003 bp, Artificial Sequence
  Synthetic
ggctccgaataagtatgttgtaactaaagtgaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataacactttagttacaacatacttattgcctcgg

SEQ ID NO: 25 - DNA, 991 bp, Artificial Sequence
  Synthetic
gatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtct

SEQ ID NO: 26 - DNA, 1053 bp, Artificial Sequence
  Synthetic
ggctccgaataagtatgttgtaactaaagtggatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtctcactttagttacaacatacttattgcctcgg

SEQ ID NO: 27 - DNA, 1191 bp, Artificial Sequence
  Synthetic
tggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgc

SEQ ID NO: 28 - DNA, 1253 bp, Artificial Sequence
  Synthetic
ggctccgaataagtatgttgtaactaaagtgtggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgccactttagttacaacatacttattgcctcgg

SEQ ID NO: 29 - DNA, 1391 bp, Artificial Sequence
  Synthetic
cgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaa

SEQ ID NO: 30 - DNA, 1453 bp, Artificial Sequence
  Synthetic
ggctccgaataagtatgttgtaactaaagtgcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaacactttagttacaacatacttattgcctcgg

SEQ ID NO: 31 - DNA, 916 bp, Artificial Sequence
  Synthetic
taatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggagg

SEQ ID NO: 32 - DNA, 978 bp, Artificial Sequence
  Synthetic
ggctccgaataagtatgttgtaactaaagtgtaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggcactttagttacaacatacttattgcctcgg

SEQ ID NO: 33 - DNA, 926 bp, Artificial Sequence
  Synthetic
cccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggagg

SEQ ID NO: 34 - DNA, 988 bp, Artificial Sequence
  Synthetic
ggctccgaataagtatgttgtaactaaagtgcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggcactttagttacaacatacttattgcctcgg

SEQ ID NO: 35 - DNA, 936 bp, Artificial Sequence
  Synthetic
agatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggagg

SEQ ID NO: 36 - DNA, 998 bp, Artificial Sequence
  Synthetic
ggctccgaataagtatgttgtaactaaagtgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggcactttagttacaacatacttattgcctcgg

SEQ ID NO: 37 - DNA, 966 bp, Artificial Sequence
  Synthetic
cggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggagg

SEQ ID NO: 38 - DNA, 1028 bp, Artificial Sequence
  Synthetic
ggctccgaataagtatgttgtaactaaagtgcggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggcactttagttacaacatacttattgcctcgg

SEQ ID NO: 39 - DNA, 850 bp, Artificial Sequence
  Synthetic
taatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataactagcataaccccttggggcctctaaacgggtcttgaggggttttttgctgaaaggagg

SEQ ID NO: 40 - DNA, 912 bp, Artificial Sequence
  Synthetic
ggctccgaataagtatgttgtaactaaagtgtaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataactagcataaccccttggggcctctaaacgggtcttgaggggttttttgctgaaaggaggcactttagttacaacatacttattgcctcgg

SEQ ID NO: 41 - DNA, 870 bp, Artificial Sequence
  Synthetic
cccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctagcataaccccttggggcctctaaacgggtcttgaggggttttttgctgaaaggagg

SEQ ID NO: 42 - DNA, 932 bp, Artificial Sequence
  Synthetic
ggctccgaataagtatgttgtaactaaagtgcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctagcataaccccttggggcctctaaacgggtcttgaggggttttttgctgaaaggaggcactttagttacaacatacttattgcctcgg

SEQ ID NO: 43 - DNA, 890 bp, Artificial Sequence
  Synthetic
agatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaactagcataaccccttggggcctctaaacgggtcttgaggggttttttgctgaaaggagg

SEQ ID NO: 44 - DNA, 952 bp, Artificial Sequence
  Synthetic
ggctccgaataagtatgttgtaactaaagtgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaactagcataaccccttggggcctctaaacgggtcttgaggggttttttgctgaaaggaggcactttagttacaacatacttattgcctcgg

SEQ ID NO: 45 - DNA, 910 bp, Artificial Sequence
  Synthetic
tagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaagctagcataaccccttggggcctctaaacgggtcttgaggggttttttgctgaaaggagg

SEQ ID NO: 46 - DNA, 972 bp, Artificial Sequence
  Synthetic
ggctccgaataagtatgttgtaactaaagtgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaagctagcataaccccttggggcctctaaacgggtcttgaggggttttttgctgaaaggaggcactttagttacaacatacttattgcctcgg

SEQ ID NO: 47 - DNA, 950 bp, Artificial Sequence
  Synthetic
cggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgcctagcataaccccttggggcctctaaacgggtcttgaggggttttttgctgaaaggagg

SEQ ID NO: 48 - DNA, 1012 bp, Artificial Sequence
  Synthetic
ggctccgaataagtatgttgtaactaaagtgcggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgcctagcataaccccttggggcctctaaacgggtcttgaggggttttttgctgaaaggaggcactttagttacaacatacttattgcctcgg

SEQ ID NO: 49 - DNA, 26 bp, Artificial Sequence
  Synthetic
cgacgctctcccttatgcgactcctg

SEQ ID NO: 50 - DNA, 25 bp, Artificial Sequence
  Synthetic
tggcgcccaacagtcccccggccac

SEQ ID NO: 51 - DNA, 23 bp, Artificial Sequence
  Synthetic
ggggcctgccaccatacccacgc

SEQ ID NO: 52 - DNA, 26 bp, Artificial Sequence
  Synthetic
aaacaagcgctcatgagcccgaagtg

SEQ ID NO: 53 - DNA, 24 bp, Artificial Sequence
  Synthetic
ggcgagcccgatcttccccatcgg

SEQ ID NO: 54 - DNA, 27 bp, Artificial Sequence
  Synthetic
gatgtcggcgatataggcgccagcaac

SEQ ID NO: 55 - DNA, 25 bp, Artificial Sequence
  Synthetic
accgcacctgtggcgccggtgatgc

SEQ ID NO: 56 - DNA, 23 bp, Artificial Sequence
  Synthetic
cggccacgatgcgtccggcgtag

SEQ ID NO: 57 - DNA, 25 bp, Artificial Sequence
  Synthetic
gcgtccggcgtagaggatcgagatc

SEQ ID NO: 58 - DNA, 24 bp, Artificial Sequence
  Synthetic
tagaggatcgagatctcgatcccg

SEQ ID NO: 59 - DNA, 27 bp, Artificial Sequence
  Synthetic
agatctcgatcccgcgaaattaatacg

SEQ ID NO: 60 - DNA, 30 bp, Artificial Sequence
  Synthetic
cccgcgaaattaatacgactcactataggg

SEQ ID NO: 61 - DNA, 25 bp, Artificial Sequence
  Synthetic
taatacgactcactatagggagacc

SEQ ID NO: 62 - DNA, 25 bp, Artificial Sequence
  Synthetic
ttagagcttgacggggaaagccggc

SEQ ID NO: 63 - DNA, 24 bp, Artificial Sequence
  Synthetic
gcgcgtaaccaccacacccgccgc

SEQ ID NO: 64 - DNA, 24 bp, Artificial Sequence
  Synthetic
cttaatgcgccgctacagggcgcg

SEQ ID NO: 65 - DNA, 27 bp, Artificial Sequence
  Synthetic
cccattcgccaatccggatatagttcc

SEQ ID NO: 66 - DNA, 29 bp, Artificial Sequence
  Synthetic
cctcctttcagcaaaaaacccctcaagac

SEQ ID NO: 67 - DNA, 26 bp, Artificial Sequence
  Synthetic
agacccgtttagaggccccaaggggt

SEQ ID NO: 68 - DNA, 27 bp, Artificial Sequence
  Synthetic
ttatgctagttattgctcagcggtggc

SEQ ID NO: 69 - DNA, 27 bp, Artificial Sequence
  Synthetic
gcagcagccaactcagcttcctttcgg

SEQ ID NO: 70 - DNA, 27 bp, Artificial Sequence
  Synthetic
actcagcttcctttcgggctttgttag

SEQ ID NO: 71 - DNA, 27 bp, Artificial Sequence
  Synthetic
ctttcgggctttgttagcagccggatc

SEQ ID NO: 72 - DNA, 33 bp, Artificial Sequence
  Synthetic
ttgttagcagccggatctcattatttataaagc

SEQ ID NO: 73 - DNA, 29 bp, Artificial Sequence
  Synthetic
ttgttagcagccggatctcattagatccc

SEQ ID NO: 74 - DNA, 29 bp, Artificial Sequence
  Synthetic
ccggatctcattatttataaagctcgtcc

SEQ ID NO: 75 - DNA, 29 bp, Artificial Sequence
  Synthetic
ccggatctcattagatcccggcggcggtc

SEQ ID NO: 76 - DNA, 31 bp, Artificial Sequence
  Synthetic
ttatttataaagctcgtccatacccccggtg

SEQ ID NO: 77 - DNA, 28 bp, Artificial Sequence
  Synthetic
ttagatcccggcggcggtcacgaactcc

SEQ ID NO: 78 - DNA, 90 bp, Artificial Sequence
  Synthetic
cctcctttcagcaaaaaacccctcaagacccgtttagaggccccaaggggttatgctagttatttataaagctcgtccatacccccggtg

SEQ ID NO: 79 - DNA, 87 bp, Artificial Sequence
  Synthetic
cctcctttcagcaaaaaacccctcaagacccgtttagaggccccaaggggttatgctagttagatcccggcggcggtcacgaactcc

SEQ ID NO: 80 - DNA, 88 bp, Artificial Sequence
  Synthetic
cctcctttcagcaaaaaacccctcaagacccgtttagaggccccaaggggttatgctagccggatctcattatttataaagctcgtcc

SEQ ID NO: 81 - DNA, 88 bp, Artificial Sequence
  Synthetic
cctcctttcagcaaaaaacccctcaagacccgtttagaggccccaaggggttatgctagccggatctcattagatcccggcggcggtc

SEQ ID NO: 82 - DNA, 92 bp, Artificial Sequence
  Synthetic
cctcctttcagcaaaaaacccctcaagacccgtttagaggccccaaggggttatgctagttgttagcagccggatctcattatttataaagc

SEQ ID NO: 83 - DNA, 88 bp, Artificial Sequence
  Synthetic
cctcctttcagcaaaaaacccctcaagacccgtttagaggccccaaggggttatgctagttgttagcagccggatctcattagatccc

SEQ ID NO: 84 - DNA, 86 bp, Artificial Sequence
  Synthetic
cctcctttcagcaaaaaacccctcaagacccgtttagaggccccaaggggttatgctagctttcgggctttgttagcagccggatc

SEQ ID NO: 85 - DNA, 86 bp, Artificial Sequence
  Synthetic
cctcctttcagcaaaaaacccctcaagacccgtttagaggccccaaggggttatgctaggcagcagccaactcagcttcctttcgg

SEQ ID NO: 86 - DNA, 57 bp, Artificial Sequence
  Synthetic
ggctccgaataagtatgttgtaactaaagtgcgacgctctcccttatgcgactcctg

SEQ ID NO: 87 - DNA, 56 bp, Artificial Sequence
  Synthetic
ggctccgaataagtatgttgtaactaaagtgtggcgcccaacagtcccccggccac

SEQ ID NO: 88 - DNA, 54 bp, Artificial Sequence
  Synthetic
ggctccgaataagtatgttgtaactaaagtgggggcctgccaccatacccacgc

SEQ ID NO: 89 - DNA, 57 bp, Artificial Sequence
  Synthetic
ggctccgaataagtatgttgtaactaaagtgaaacaagcgctcatgagcccgaagtg

SEQ ID NO: 90 - DNA, 55 bp, Artificial Sequence
  Synthetic
ggctccgaataagtatgttgtaactaaagtgggcgagcccgatcttccccatcgg

SEQ ID NO: 91 - DNA, 58 bp, Artificial Sequence
  Synthetic
ggctccgaataagtatgttgtaactaaagtggatgtcggcgatataggcgccagcaac

SEQ ID NO: 92 - DNA, 56 bp, Artificial Sequence
  Synthetic
ggctccgaataagtatgttgtaactaaagtgaccgcacctgtggcgccggtgatgc

SEQ ID NO: 93 - DNA, 54 bp, Artificial Sequence
  Synthetic
ggctccgaataagtatgttgtaactaaagtgcggccacgatgcgtccggcgtag

SEQ ID NO: 94 - DNA, 56 bp, Artificial Sequence
  Synthetic
ggctccgaataagtatgttgtaactaaagtggcgtccggcgtagaggatcgagatc

SEQ ID NO: 95 - DNA, 55 bp, Artificial Sequence
  Synthetic
ggctccgaataagtatgttgtaactaaagtgtagaggatcgagatctcgatcccg

SEQ ID NO: 96 - DNA, 58 bp, Artificial Sequence
  Synthetic
ggctccgaataagtatgttgtaactaaagtgagatctcgatcccgcgaaattaatacg

SEQ ID NO: 97 - DNA, 61 bp, Artificial Sequence
  Synthetic
ggctccgaataagtatgttgtaactaaagtgcccgcgaaattaatacgactcactataggg

SEQ ID NO: 98 - DNA, 56 bp, Artificial Sequence
  Synthetic
ggctccgaataagtatgttgtaactaaagtgtaatacgactcactatagggagacc

SEQ ID NO: 99 - DNA, 56 bp, Artificial Sequence
  Synthetic
ccgaggcaataagtatgttgtaactaaagtgttagagcttgacggggaaagccggc

SEQ ID NO: 100 - DNA, 55 bp, Artificial Sequence
  Synthetic
ccgaggcaataagtatgttgtaactaaagtggcgcgtaaccaccacacccgccgc

SEQ ID NO: 101 - DNA, 55 bp, Artificial Sequence
  Synthetic
ccgaggcaataagtatgttgtaactaaagtgcttaatgcgccgctacagggcgcg

SEQ ID NO: 102 - DNA, 58 bp, Artificial Sequence
  Synthetic
ccgaggcaataagtatgttgtaactaaagtgcccattcgccaatccggatatagttcc

SEQ ID NO: 103 - DNA, 60 bp, Artificial Sequence
  Synthetic
ccgaggcaataagtatgttgtaactaaagtgcctcctttcagcaaaaaacccctcaagac

SEQ ID NO: 104 - DNA, 57 bp, Artificial Sequence
  Synthetic
ccgaggcaataagtatgttgtaactaaagtgagacccgtttagaggccccaaggggt

SEQ ID NO: 105 - DNA, 58 bp, Artificial Sequence
  Synthetic
ccgaggcaataagtatgttgtaactaaagtgttatgctagttattgctcagcggtggc

SEQ ID NO: 106 - DNA, 58 bp, Artificial Sequence
  Synthetic
ccgaggcaataagtatgttgtaactaaagtggcagcagccaactcagcttcctttcgg

SEQ ID NO: 107 - DNA, 58 bp, Artificial Sequence
  Synthetic
ccgaggcaataagtatgttgtaactaaagtgactcagcttcctttcgggctttgttag

SEQ ID NO: 108 - DNA, 58 bp, Artificial Sequence
  Synthetic
ccgaggcaataagtatgttgtaactaaagtgctttcgggctttgttagcagccggatc

SEQ ID NO: 109 - DNA, 64 bp, Artificial Sequence
  Synthetic
ccgaggcaataagtatgttgtaactaaagtgttgttagcagccggatctcattatttataaagc

SEQ ID NO: 110 - DNA, 60 bp, Artificial Sequence
  Synthetic
ccgaggcaataagtatgttgtaactaaagtgttgttagcagccggatctcattagatccc

SEQ ID NO: 111 - DNA, 60 bp, Artificial Sequence
  Synthetic
ccgaggcaataagtatgttgtaactaaagtgccggatctcattatttataaagctcgtcc

SEQ ID NO: 112 - DNA, 60 bp, Artificial Sequence
  Synthetic
ccgaggcaataagtatgttgtaactaaagtgccggatctcattagatcccggcggcggtc

SEQ ID NO: 113 - DNA, 62 bp, Artificial Sequence
  Synthetic
ccgaggcaataagtatgttgtaactaaagtgttatttataaagctcgtccatacccccggtg

SEQ ID NO: 114 - DNA, 59 bp, Artificial Sequence
  Synthetic
ccgaggcaataagtatgttgtaactaaagtgttagatcccggcggcggtcacgaactcc

SEQ ID NO: 115 - DNA, 121 bp, Artificial Sequence
  Synthetic
ccgaggcaataagtatgttgtaactaaagtgcctcctttcagcaaaaaacccctcaagacccgtttagaggccccaaggggttatgctagttatttataaagctcgtccatacccccggtg

SEQ ID NO: 116 - DNA, 118 bp, Artificial Sequence
  Synthetic
ccgaggcaataagtatgttgtaactaaagtgcctcctttcagcaaaaaacccctcaagacccgtttagaggccccaaggggttatgctagttagatcccggcggcggtcacgaactcc

SEQ ID NO: 117 - DNA, 119 bp, Artificial Sequence
  Synthetic
ccgaggcaataagtatgttgtaactaaagtgcctcctttcagcaaaaaacccctcaagacccgtttagaggccccaaggggttatgctagccggatctcattatttataaagctcgtcc

SEQ ID NO: 118 - DNA, 119 bp, Artificial Sequence
  Synthetic
ccgaggcaataagtatgttgtaactaaagtgcctcctttcagcaaaaaacccctcaagacccgtttagaggccccaaggggttatgctagccggatctcattagatcccggcggcggtc

SEQ ID NO: 119 - DNA, 123 bp, Artificial Sequence
  Synthetic
ccgaggcaataagtatgttgtaactaaagtgcctcctttcagcaaaaaacccctcaagacccgtttagaggccccaaggggttatgctagttgttagcagccggatctcattatttataaagc

SEQ ID NO: 120 - DNA, 119 bp, Artificial Sequence
  Synthetic
ccgaggcaataagtatgttgtaactaaagtgcctcctttcagcaaaaaacccctcaagacccgtttagaggccccaaggggttatgctagttgttagcagccggatctcattagatccc

SEQ ID NO: 121 - DNA, 117 bp, Artificial Sequence
  Synthetic
ccgaggcaataagtatgttgtaactaaagtgcctcctttcagcaaaaaacccctcaagacccgtttagaggccccaaggggttatgctagctttcgggctttgttagcagccggatc

SEQ ID NO: 122 - DNA, 117 bp, Artificial Sequence
  Synthetic
ccgaggcaataagtatgttgtaactaaagtgcctcctttcagcaaaaaacccctcaagacccgtttagaggccccaaggggttatgctaggcagcagccaactcagcttcctttcgg

SEQ ID NO: 123 - DNA, 1410 bp, Artificial Sequence
  Synthetic
tccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctcgatcccgcgaaattaatacgactcactatagggagaccacaacggtttccctctagaaataattttgtttaactttaagaaggagatatacatatggtctcaaagggggaagaggataatatggcgatcatcaaggagtttatgcgtttcaaagttcatatggaaggcagtgttaacggccacgagttcgagattgagggggagggggaaggacgcccttacgaagggacccaaacagctaaattaaaagtgactaaagggggtccgttgcctttcgcgtgggatatcttatcaccgcagttcatgtatggatcgaaggcttatgtaaaacaccccgcagacatccctgattaccttaaactttcattcccggaagggtttaaatgggagcgtgtaatgaattttgaggacggaggggtagttacggtcactcaagatagcagtttacaagatggagagttcatttataaggtgaagttacgtggaacaaacttcccgtcagacggtcccgttatgcaaaaaaagacgatgggctgggaggcttcttccgagcgcatgtatccagaagacggtgcactgaaaggcgagatcaagcaacgcctgaaattaaaggacgggggacattatgacgccgaggtcaaaactacatacaaagctaagaaacccgttcagttaccaggtgcctacaacgtaaacattaaattggacattacgtcgcataacgaggattacacgatcgtggaacaatatgaacgtgctgagggtcgccactccaccgggggtatggacgagctttataaataatgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggc

SEQ ID NO: 124 - DNA, 1410 bp, Artificial Sequence
  Synthetic
gcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccaatccggatatagttcctcctttcagcaaaaaacccctcaagacccgtttagaggccccaaggggttatgctagttattgctcagcggtggcagcagccaactcagcttcctttcgggctttgttagcagccggatctcattatttataaagctcgtccatacccccggtggagtggcgaccctcagcacgttcatattgttccacgatcgtgtaatcctcgttatgcgacgtaatgtccaatttaatgtttacgttgtaggcacctggtaactgaacgggtttcttagctttgtatgtagttttgacctcggcgtcataatgtcccccgtcctttaatttcaggcgttgcttgatctcgcctttcagtgcaccgtcttctggatacatgcgctcggaagaagcctcccagcccatcgtctttttttgcataacgggaccgtctgacgggaagtttgttccacgtaacttcaccttataaatgaactctccatcttgtaaactgctatcttgagtgaccgtaactacccctccgtcctcaaaattcattacacgctcccatttaaacccttccgggaatgaaagtttaaggtaatcagggatgtctgcggggtgttttacataagccttcgatccatacatgaactgcggtgataagatatcccacgcgaaaggcaacggaccccctttagtcacttttaatttagctgtttgggtcccttcgtaagggcgtccttccccctccccctcaatctcgaactcgtggccgttaacactgccttccatatgaactttgaaacgcataaactccttgatgatcgccatattatcctcttccccctttgagaccatatgtatatctccttcttaaagttaaacaaaattatttctagagggaaaccgttgtggtctccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccgga